(19) United States
(12) Patent Application Publication
(10) Pub. No.: US 2005/0202421 A1
(43) Pub. Date: Sep. 15, 2005

(54) Method for diagnosis and treatment of rheumatoid arthritis

(76) Inventors: Raphael Hirsch, Pittsburgh, PA (US); Sherry L. Thornton, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 10/287,436

(22) Filed: Oct. 31, 2002

Related U.S. Application Data
(60) Provisional application No. 60/336,220, filed on Oct. 31, 2001.

(51) Int. Cl. C12Q 1/68

(52) U.S. Cl. 435/6

(57) ABSTRACT

The onset and progression of chronic autoimmune diseases, including human rheumatoid arthritis (RA) are likely determined by differential expression of genes that influence inflammatory and immune responses. The collagen-induced arthritis (CIA) mouse model for RA exhibits many of the same genetic and immunological features of RA; however, the profiles of gene expression during the inflammatory and immune responses of CIA or RA have not been well characterized. Previous studies have demonstrated that mRNA levels, particularly that of cytokines, can change over the course of CIA. To determine the contribution of various genes in the pathogenesis of CIA, microarray technology was used to simultaneously monitor 8,734 target cDNAs to discover arthritic stage-specific genes. The resulting gene expression profile identified 333 genes that were at least 2-fold up-regulated in all synovial samples: normal, acute disease and chronic disease. In addition, 385 disease-specific genes were identified that were greater than or equal to 2-fold over- or under-expressed in the disease state as compared to normal synovium. Clustering analysis among the arthritic states allowed for the identification of four distinct kinetic expression patterns based on differential expression levels in normal, acute disease and chronic disease synovial samples.

### CROSS REFERENCE TO RELATED APPLICATIONS

This application claims the benefit of provisional application Ser. No. 60/336,220, filed Oct. 31, 2001, the disclosure of which is incorporated by reference herein in its entirety.

### GOVERNMENT INTEREST IN THE INVENTION

Certain aspects of the invention disclosed herein were made with United States government support under National Institutes of Health grants AI34958, AR44059, AR47712, and AR42632. The United States government has certain rights in these aspects of the invention.

### INCORPORATION-BY-REFERENCE OF CD-ROM DATA

Applicants hereby incorporate by reference in their entirety two copies of a compact disc, labeled “Copy 1” and “Copy 2,” respectively, containing table1.1.txt, 2,276,363 size in bytes, created on Oct. 31, 2002; table1.2.txt, 1,335,492 size in bytes, created on Oct. 31, 2002; table1.3.txt, 2,924,772 size in bytes, created on Oct. 31, 2002; table2.1.txt, 817,381 size in bytes, created on Oct. 31, 2002; table2.2.txt, 1,003,344 size in bytes, created on Oct. 31, 2002; and table2.3.txt, 604,772 size in bytes, created on Oct. 31, 2002.

### BACKGROUND OF THE INVENTION

1. Field of the Invention

The invention relates generally to materials and methods for diagnosis and treatment of rheumatoid arthritis (RA) and related conditions. More specifically, the invention relates to nucleic acids, proteins, arrays thereof, methods for diagnosis and methods for analyzing the severity of RA and related conditions using, for example, patterns of up- and down-regulation of specific genes identified by microarray technology. The invention further relates to the treatment of RA by activating those genes or proteins that are down-regulated and/or inhibiting those genes or proteins that are up-regulated. The invention also relates to identifying and using targets for drug treatment, methods of screening candidate drugs, and methods for identifying optimal treatment approaches for a specific patient.

2. DESCRIPTION OF THE RELATED ART

Collagen-induced arthritis (CIA) in mice has been utilized to study underlying mechanisms of autoimmune arthritis because of its clinical, histologic, immunologic and genetic similarity to rheumatoid arthritis (RA). Although several immunoregulatory genes have been implicated in this model system, molecular mechanisms underlying the pathophysiology have only been partially defined.

In CIA, progression of disease is associated with changes in the cell types infiltrating the joint. The acute phase of the disease is characterized by a predominantly neutrophilic infiltrate, with monocytes and lymphocytes constituting approximately 5% of the inflammatory cell population. By day 49, a decrease in lymphocytes is observed, with an increase in fibroblast/macrophage type cells and an increasingly fibrotic appearance. In conjunction with the changes of cellular infiltrate, mRNA and protein expression levels of several cytokines and chemokines also change over the course of disease. For example, TNFα protein expression in the joint precedes that of IL-1β and IFN-γ is expressed shortly after disease onset, but not late in disease. IL-1β and IL-10 mRNAs, but not those of IFN-γ and IL-5, are detected in late disease.

Classical approaches to studying inflammatory mediators in arthritis have focused on identifying and analyzing these mediators individually. While this method has proven extremely productive, arthritis represents a complex and multifactorial pathophysiology that likely involves hundreds or thousands of individual gene products acting in concert. Improved understanding of the genes that are operative during the development of the inflammatory lesion may aid in the design of disease-specific therapies. Several methods to examine coordinated gene expression have been developed, including Northern blot, ribonuclease protection assay (RPA), differential display and sequencing of cDNA libraries and expressed sequence tags (ESTs). Using total paw RNA from a mouse with CIA and using the method of RPA, the inventors have previously demonstrated distinct changes in mRNA expression of a number of cytokines in early and late CIA. IL-2, IL-6, MIP2 and IL-1β were found predominantly in early disease, whereas, TGFβ was found predominantly in late disease. IL-11, IL-1ra, MIP1α, RANTES, TNFα and TNFβ were present both in early and late disease. These changes in gene expression within the joint likely affect the disease pathology observed at the cellular and macroscopic level. Whether a similar temporal change in cellular infiltrate and mRNA expression profiles also occurs in RA is not clear, as few synovial biopsies have been performed at the very early stages of RA. However, since most of the previously mentioned cytokines are found in synovial fluids and chronic RA synovium, these findings have relevance to RA.

The recent advent of high-throughput methods, such as serial analysis of gene expression (SAGE) and DNA microarrays, have allowed large-scale, genome-wide characterizations of gene expression to be performed. Whole-genome expression profiling represents a major advance in genome-wide functional analysis. In a single assay, the transcriptional response of each gene to a change in cellular state, including a disease or a chemical perturbation, can be measured. These changes in gene expression can reflect changes in mRNA levels or changes in the cells (proliferation or infiltration) that synthesize these mRNAs. DNA microarray technology is well-suited for analyzing chronic diseases, such as autoimmune arthritis, because of the wide spectrum of genes and endogenous mediators involved. A recent report describing the analysis of RA and inflammatory bowel disease tissues used a microarray of about 100 genes known to have a role in inflammation. IL-6 and several matrix metalloproteinases were markedly upregulated in RA tissues; however the observed upregulation of matrix metallo-elastase (HME) was unexpected, since its expression was previously thought to be limited to alveolar macrophages and placental cells. Analyses such as these are able to identify genes, both known and novel, and discover their coordinately regulated expression during the disease process.

Analysis of global gene expression in disease joints is likely to lead to a fuller understanding of the inflammatory processes responsible for arthritis. In the present study, DNA microarray technology was used to identify novel genes and biological pathways involved in CIA and to test the hypothesis that the previously observed set of stage-specific differentially activated genes in CIA represents a larger transcriptional profile.

### SUMMARY OF THE INVENTION

Using microarray analysis, the expression of 8734 cDNAs was analyzed during various stages of mouse collagen induced arthritis (CIA), an animal model of RA. From the results, a method for the diagnosis and treatment of RA was developed.

Embodiments relate to methods for the diagnosis and analysis of autoimmune disease or arthritide, in a patient. The methods can include, for example, obtaining a patient sample containing mRNA; analyzing gene expression using the mRNA that results in a gene expression signature of that mRNA, wherein the gene expression signature includes the identification and quantitation of gene expression from genes that have been identified as being differentially expressed in RA; and using that gene expression signature to diagnose or analyze the autoimmune disease or arthritide in said patient, wherein said gene expression of at least about 60% of said genes correlates with that of said gene signature.

The autoimmune disease or arthritides can be, for example, Rheumatoid Arthritis, Lupus, Ankylosing Spondylitis, fibrositis, fibromyalgia, osteoarthritis, Gout, Juvenile Rheumatoid Arthritis, an autoimmune disease caused by an infectious agent, and the like. Preferably, the autoimmune disease or arthritide can be rheumatoid arthritis. The patient can be, for example, a human, a primate, a dog, a cat, a horse, a sheep, and the like.

The analysis can be, for example, an analysis of severity of the disease, an analysis of pain manifestation, an analysis of deformity, an analysis of treatment methods, an analysis of treatment efficacy, and the like.

The gene expression analysis can involve at least about 10 genes that are identified as differentially expressed in arthritis, preferably at least about 50 genes that are identified as differentially expressed in arthritis, more preferably at least about 100 genes that are identified as differentially expressed in arthritis, and the like.

The genes identified can be expressed at least about 1.5 fold higher or lower than normal, at least about 2 fold higher or lower than normal, at least about 3 fold higher or lower than normal, and the like.

The genes can include, for example, the 385 genes or ESTs in Table 1 (SEQ ID NOS:1-385), homologs, variant thereof, and the like. The genes can include the genes in cluster A, and in embodiments the genes in cluster A can be down-regulated (SEQ ID NOS:1-37) at least about 2 fold, for example. Further, the genes can include the genes in cluster B, and in embodiments the genes in cluster B can be up-regulated (SEQ ID NOS:1-37) at least about 2 fold only in late or severe disease, for example. The genes can include the genes in cluster C, and in embodiments the genes in cluster C can be up-regulated (SEQ ID NOS:1-37) at least about 2 fold only in early or mild disease, for example. Also, the genes can include the genes in cluster D, and in embodiments the genes in cluster D can be up-regulated (SEQ ID NOS:1-37) at least about 2 fold in early or mild disease and more in late or severe disease, for example. Furthermore, genes can include the genes in cluster E, and in embodiments the genes in cluster E can be up-regulated (SEQ ID NOS:1-37) at least about 2 fold in both early or mile and late or severe disease, for example.

Also, the differentially expressed genes can include the 385 genes identified as SEQ ID NOS:1-385, for example. If the genes in clusters B or D are upregulated, the disease can be diagnosed as severe. Furthermore, if the genes in cluster A are upregulated, the disease can be diagnosed as moderate to low-grade.

Further, the gene expression of at least about 70% of the genes correlates with that of the gene signature, preferably, the gene expression of at least about 80% of the genes correlates with that of the gene signature, more preferably, the gene expression of at least about 90% of the genes correlates with that of the gene signature, still more preferably, the gene expression of at least about 95% of the genes correlates with that of the gene signature, and the like.

Aspects and embodiments of the invention further provide methods for the treatment of RA that include down-regulating at least one of the genes identified in clusters B through D. Such down-regulation can be achieved by adding antisense oligonucleotides specific for the gene that is being down-regulated, or by adding or expressing a repressor of the gene that is being down-regulated.

In other embodiments, the invention provides methods for the treatment of RA which involve up-regulating at least one of the genes in cluster A, for example, by adding or expressing a transcriptional activator of the gene that is being up-regulated, or by adding a vector that expresses the protein encoded by the gene that is being up-regulated.

Further aspects and embodimetns of the invention provide methods for the identification of genes for targeting in the treatment of rheumatoid arthritis in a mammal other than a mouse, which methods involve identifying homologs of SEQ ID NOS:1-385.

Still other aspects and embodimetns of the invention include methods for the diagnosis of rheumatoid arthritis in a mammal, the methods including obtaining a tissue or fluid sample from a diseased patient; isolating mRNA from said sample; using the isolated mRNA to analyze the gene expression of at least about 40 genes, selected from the group consisting of SEQ ID NOS:1-385 or a homolog thereof, obtaining a fingerprint of the patient's gene expression; and identifying whether at least about 60% of said fingerprint is at least about 2 fold differentially expressed from that of a normal patient.

Other embodiments include an array or a genechip, specific for rheumatoid arthritis, including at least 10 of the genes selected from the group consisting of SEQ ID NOS:1-385 or homologs thereof. The array or genechip can include at least 40, 50, 75, 100, or more, of the genes selected from the group consisting of SEQ ID NOS:1-385 or homologs thereof. In some embodiments, the array or genechip consists essentially of such genes, including up to all of the genes of SEQ ID NOS:1-385 or homologs thereof. Such genes can allow for the identification of the severity of the disease, the prognosis of the disease, the diagnosis of the disease, the most efficacious treatment of the disease in a specific patient, and the like.

In other embodiments, the invention provides methods for the diagnosis or analyses of autoimmune disease or rheumatoid arthritis, including: obtaining mRNA from a patient; using the mRNA as a probe for the analysis of the arrays or genechips disclosed herein; and comparing the results obtained with those of a normal patient.

Additional embodiments and aspects provide methods of screening the efficacy of a candidate drug in vitro for the treatment of collagen-induced arthritis including: identifying vascular endothelial cells expressing FARP mRNA and protein; introducing a candidate drug to said endothelial cells; and evaluating whether said candidate drug causes enhanced or normalized apoptosis of vascular endothelial cells.

Further, the invention in some embodiments provides methods and materials for reducing the symptoms associated with collagen-induced arthritis including: identifying a subject suffering from collagen-induced arthritis; and administering a compound effective to deplete at least one of the group of FARP mRNA, FARP protein, FARP receptor binding, and FARP activity. Such compound can include, for example, an anti-FARP antibody, capable of interfering with binding of FARP to a FARP receptor.

### BRIEF DESCRIPTION OF THE DRAWINGS

FIG. 1. Hierarchical cluster analysis of 385 genes differentially expressed during CIA. The left panel shows the distribution of gene expression across the hierarchical tree structure in which the values for the first normal sample (1) are set to 1. Rows represent individual genes; columns represent individual values of duplicate samples for each experimental time point. Each cell in the matrix represents the expression level of a single transcript with red and green indicating transcript levels above and below the normal values for that gene across all samples, respectively. The color code for the signal strength in the classification scheme is shown in the box at the bottom left of the panel. Color intensity from pale to deep indicates trust values for the expression of each specific transcript. The colored side bar indicates the five basic clusters of gene expression, with letters corresponding to their grouping. The mean values of all the genes within the indicated groups (A-E) are graphed on the right.

FIG. 2. Comparison of microarray and RT-PCR analyses of representative genes in CIA. The patterns of IL-2Rγ and follistatin-like gene mRNA levels, determined by DNA microarray analysis from pooled RNA, are compared to patterns determined by real time RT-PCR analysis of two individual RNA samples.

FIG. 3. IL2-Rγ is expressed in the synovial tissue during collagen-induced arthritis. Panel A (dark-field illumination) and panel B (bright-field illumination) show a section through the joint from a normal mouse paw. There is no signal in the joint tissue or surrounding periosteal tissue. Panels C and D show a section through a CIA mouse paw 28 days following primary CII immunization. There is positive signal (bright white grains) in the synovial tissue (arrow) indicating the presence of RNA transcripts for IL2-Rγ. Panels E and F represent a section through the paw of a CIA mouse 49 days following primary CII immunization. There is an extensive chronic inflammatory reaction in the tissue (*) surrounding the cortical bone tissue. Despite the chronic inflammatory reaction in the tissue no significant IL2-Rγ is present in the lesion late in disease. (PO periosteal; CB cortical bone; SY synovium; AS articular surface; Mag 100×)

FIG. 4. Tissue-specific expression of differentially regulated genes in lymphoid organs and cells. The presence of specific gene sequences in cDNA libraries generated from the indicated tissues was obtained from the NCBI database using the LocusLink and Unigene databases.

FIG. 5. Classification of selected annotated genes. Bars indicate the number of the characterized genes that are involved in the specified biological function (A) or pathway (B). The number of genes in each of the five expression patterns is indicated on each bar. Some genes are represented in more than one category.

### BRIEF DESCRIPTION OF TABLES 1 AND 2

As mentioned above, filed herewith on two compact discs are two copies of Table 1, including Tables 1.1-1.3, and Table 2, including Tables 2.1-2.3. The compact discs are labeled as “Copy 1” and “Copy 2.” Each disc has identical content. The contents of the discs are hereby incorporated by reference in their entireties.

Table 1 Listing of mouse gene accession numbers, mouse gene name, human mRNA homolog, human protein homologs, and Genbank source of human homolog information. These genes are divided into clusters A through E by expression characteristics as explained herein. Human homologs were identified using unigene and homologene functions at the NCBI database. Further information on the homologous human mRNA sequences can be found in Table 1.1 under the accession number of interest. Similarly, further information on the homologous human protein sequences can be found in Table 1.2, and further information on the “Genbank source” can be found in Table 1.3.

Table 2 Listing of relevant ESTs. The ESTs are grouped into clusters A through E, as explained herein. Listed are the name of the gene (if known), the accession number of the corresponding homologous human mRNA (if known), the Genbank source number of the human mRNA information, the Genbank accession number for the mouse gene, and a description of similar genes, if known. Further information on the homologous human mRNA sequences corresponding to the ESTs can be found in Table 2.1, under the accession number of interest. Similarly, further information relating to the Genbank source number (human) can be found in Table 2.2, and information corresponding to the Genbank accession numbers (mouse) can be found in Table 2.3.

### DETAILED DESCRIPTION OF THE PREFERRED EMBODIMENT

Using microarray analysis, the expression of 8734 cDNAs was analyzed during various stages of mouse CIA, an animal model of RA. From the results, a method for the diagnosis and treatment of RA was developed. Of the 8,734 genes analyzed, 330 were induced and 55 were down-regulated greater than two-fold in early or late diseased paws, as compared to normal paws. Hierarchical clustering resulted in five distinct expression patterns that correlated with histopathologic changes in the paw. Of the 385 genes, the identities of 240 are known. These genes are biologically classifiable into 19 functional categories, the largest being immunity and defense, and into 20 pathway categories, including membrane, secreted and extracellular. Of the known genes, the majority have not been described as playing a role in arthritis. Many of these genes are involved in cell proliferation, differentiation, tumorigenesis, apoptosis, and inflammation. Thus, these global gene expression patterns in diseased paws reveal a large number of genes novel to arthritis, and distinct gene expression profiles distinguishing early and late CIA whose further characterization will advance the understanding of the basic mechanisms responsible for arthritis.

The results of the analysis of the mouse model of RA include a set of differentially expressed genes that can be used for a variety of purposes. The set of differentially expressed genes can be thought of as a “signature” or a “fingerprint” of RA. Thus, some embodiments of the present invention include DNA arrays or genechips that include one or more of the differentially expressed mouse or human genes identified herein. Further embodiments can include a specific subset of the differentially expressed genes that can represent, for example, genes that are only up-regulated in late disease or genes that are only up-regulated in early disease. A “human Rheumatoid Arthritis genechip” can be used to further study the gene expression of RA as well as other auto-immune diseases, in animal models or in human patients.

The results of the analysis of the mouse model of RA are also useful in identifying and developing various embodiments of a “human Rheumatoid Arthritis genechip” which includes human homologs of the mouse genes identified herein as well as independently identified genes. The chip and the information obtained can be used to develop methods for diagnosis, prognosis, and analysis of the efficacy of treatments.

The analysis of mouse genes herein is believed to have covered approximately one third of the genes typically expressed in the mouse genome (a comparable number to that expressed in the human genome). Thus, one embodiment is a method for the identification of other mouse genes involved in RA. In order to thoroughly identify the genes that are differentially expressed in the mouse, arrays or genechips that include a thorough representation of mouse mRNAs are analyzed using the same method of analysis that identified the RA-specific genes identified herein. However, using the genes identified in the initial analysis of 8734 genes, human or other mammalian homologs can be identified and the differential expression confirmed. The method is also useful for further identifying genes that are up- and down-regulated in human or other mammalian RA and related conditions. Numerous human homologs of the mouse genes are also differentially regulated in human RA comparably to the differential regulation in mouse CIA.

Thus a method is described herein that identifies the pattern of specific differentially expressed genes, also referred to as the “signature” or “fingerprint” for a particular disease state or a particular patient. The signature is used to diagnose RA in a patient and to analyze the severity of the disease. The pattern of specifically up and down-regulated genes is compared to a “normal” patient, a patient who does not have RA.

Briefly, genes that are differentially regulated from the normal in patients with RA are identified by any method known to one of skill in the art. With identification of genes involved in the disease and progression of RA, the genetic data are useful in developing a number of methods for use on a patient who has or may have RA or other arthritides.

Preferred methods involve the identification of the signature of differential expression of one or more of the identified genes for a specific patient. In some embodiments, the method includes isolation of mRNA from a diseased tissue, blood sample, or synovial fluid sample from a patient. The expression of the genes that are specifically identified as differentially regulated is analyzed. The “signature” is produced as the pattern of up and down-regulated genes within that patient's sample. The signature can be used for diagnostic methods, for prognostic methods, for analysis of the most efficacious treatment for the patient, and for analysis of the efficacy of the treatment or the progression of the disease.

Identifying Human Genes that are Differentially Regulated in RA

In some embodiments, the genes that are differentially regulated in human RA are identified by a) using mouse genes associated with CIA to identify human and/or other mammalian homologs thereof using database comparisons, b) using mouse genes associated with CIA to isolate homologs from gene libraries of an animal of interest and/or c) using genes that are known to be involved in mammalian RA and mammalian homologs of those genes.

In a further embodiment, the genes that are differentially regulated in mammalian RA are identified by microarray analysis using mRNAs from a mammal with RA, using a method comparable to that used herein for identification of the mouse genes. Preferably, the methods identify a thorough representation of the genes involved in RA by one method or another.

In some embodiments, the mRNAs from the mammal with RA are obtained from a tissue, biological fluid or mixture thereof that contains mRNA. In further embodiments, the mRNAs are isolated from diseased synovial tissue or synovial fluid. In still further embodiments, the mRNAs are isolated from a blood sample, a saliva sample, or a urine sample. In preferred embodiments, a patient sample is used for which the expression of genes is altered due to the disease.

Homologs can be genes or DNAs that are 40% similar or more to the mouse genes identified, alternatively, the homologs are at least 50% similar, including 55% similar, 60% similar, 65% similar, 70% similar, 75% similar, 80% similar, 85% similar, 90% similar, 95% similar, and 99% similar. Homologs that are more similar are generally most closely related to the mouse sequence, and thus are in many cases most likely to exhibit similar differential expression in RA. However, the amount of similarity can vary depending on the importance of the region of the gene identified. For example, if the mouse gene is a kinase, the kinase regions are likely to be more homologous or similar then the other regions. The homologs can be DNAs that hybridize under stringent conditions to the mouse genes identified. The stringent conditions under which a homologous gene or DNA will hybridize with the mouse gene can be defined as follows: 0.1×SSPE, 0.1% SDS wash solution at 65° C. with 2 washes. (1×SSPE is 180 mM NaCl, 10 mM NaH₂PO₄, 1 mM EDTA (pH 7.4)). The identification of mammalian homologs can be accomplished using any method known to one of skill in the art. Any genes that have been identified or will be identified as being involved in the disease can be included. Certain genes having a more central or “important” role in different aspects of the disease are thus identifiable. Thus, the subset of genes that are analyzed or contained in a microarray or genechip can be chosen based on the direct or indirect role the gene is found to play in the disease. Alternatively, subsets can be chosen based on what aspect of the disease is being tested. Thus, in some embodiments, those genes that are identified as being involved in “activating” the disease will be included particularly when diagnosis is the desired result. In a further embodiment, those genes that are identified as involved in “progression” of the disease will be included, particularly when treatment, prognosis, or staging of disease is being analyzed. In a further embodiment, those genes involved in remission, regression, or healing of the disease are included, particularly when prognosis, efficacy of treatment, and/or staging of the disease are being analyzed.

The above method can be altered and applied to all mammals. Thus, in some embodiments, the patient is a mammal. In a further embodiment, the mammal is a human, primate, dog, cat, or horse. Because the incidence of RA in humans is particularly significant, some embodiments include methods for the diagnosis, prognosis and analysis of human RA. Human homologs are identified by methods known to those of skill in the art. In one embodiment, human homologs are identified using computer programs that search for “closest homologs” by inputting the mouse genes and ESTs identified herein. In a further embodiment, the computer analysis can use “active” portions of the sequences or those parts of the gene sequences that are known to be more highly conserved between mammals. The portions that are more highly conserved can be involved in the activity of the protein expressed therefrom. A variety of computer programs can be used to identify the closest mammalian homologs. In many cases, there can be more than one human homolog that corresponds to the mouse gene.

In a further embodiment, human homologs are identified by performing the microarray analysis that was used to identify the mouse genes herein. In preferred embodiments, a thorough representation of the human genes that are expressed is analyzed. For example, it is believed that approximately 100,000 genes are actively expressed or included in the human genome. Thus, in order to thoroughly identify those that are involved in the disease RA, a complete representation of the approximately 100,000 genes are analyzed. For example, one or more arrays that contain a thorough representation of the human genome are used to analyze gene expression. In one embodiment, the arrays are from one or more tissues or fluids. In a further embodiment, the arrays are analyzed in duplicate, in triplicate, or in multiple copies. In one embodiment, differential expression can be identified as at least about a 1.4 to 2 fold difference in expression from normal. In a further embodiment, the differential expression is identified as about a 1.6 to 2 fold difference in expression. In a further embodiment, the genes are identified as differentially expressed in RA when there is at least about a 2 fold difference in expression from normal. In a further embodiment, the genes are identified as differentially expressed in RA when there is at least about a 2.3 fold difference in expression from normal. In a further embodiment, the genes are identified as differentially expressed in RA when there is at least about a 2.5 fold difference in expression from normal, including at least about 2.6 fold, 2.7 fold, 2.8 fold, 2.9 fold, 3 fold, 3.5 fold, 4 fold, and 5 fold. However, some genes can show a higher difference in expression than others. These genes can be more involved or alternatively, equally involved in the manifestation of disease as a gene that is less differentially expressed.

From the above analysis, a “signature” or “fingerprint” can be produced that includes the genes that are differentially expressed in the disease and the range of expression that can be seen among different patients. In one embodiment, the differential expression can be due to different aspects and manifestations of the disease. For example, the fingerprint can be a fingerprint of early RA, late RA, mild RA, extreme RA, RA in remission, a manifestation of RA with little pain, but considerable deformity, a manifestation of RA with considerable pain, but little deformity, etc.

The expression of many of the genes identified is confirmed using alternative methods known to one of skill in the art, including Northern blotting, quantitative PCR techniques such as real-time PCR, or other methods of expression analysis. Alternatively, the translation products and expression can be analyzed by methods known to one of skill in the art, such as Western blotting, activity assays, etc.

In a further embodiment, the genes identified as part of the “signature” or “fingerprint” are further analyzed as to their involvement in the disease. In one embodiment, a gene is further analyzed by any method known to one of skill in the art and can identify the involvement in activation, progression, pain manifestation, deformation, and treatment of the disease. Patients that express certain genes or subsets identified above will often show a greater response to certain types of treatments then others. For example, if one patient expresses high amounts of IL-2, that patient would respond better to treatments that target IL-2 activity, expression, or the downstream effects of IL-2.

One embodiment of this “signature” or “fingerprint” is an array or a genechip that includes the genes that are identified as differentially expressed in one or all manifestations of RA, which can be referred to as a “human Rheumatoid Arthritis genechip.” A variety of genechips can be produced that are specific to different aspects of the disease. In one embodiment, a genechip can be produced with only those genes that are identified as possessing key roles in each aspect of the disease. In a further embodiment, a genechip can be produced that includes only those genes that are expressed late in disease or in severe disease.

Method of Diagnosis Prognosis, and Treatment Analysis of a Patient with Rheumatoid Arthritis

The genes that are identified above as being involved in RA can be analyzed as to differential expression in a specific patient by any means known to one of skill in the art. Some embodiments involve isolation of the mRNA from a patient sample.

Briefly, mRNA is isolated from at least one tissue or sample from the patient. In one embodiment, the sample is a diseased tissue sample, including but not limited to synovial tissue. In a further embodiment, the sample is a fluid containing disease cells or mRNA, including, but not limited to, synovial fluid, and blood.

The mRNA can then be used to analyze gene expression by any method known to one of skill in the art. In one embodiment, the mRNA is used to analyze a “human Rheumatoid Arthritis genechip” or array. From this analysis, a specific patient “signature” of the genes and amount of differential expression is produced. The amount of differential expression is compared to a normal patient. In one embodiment, the ranges and values of expression for a normal patient are derived using at least 2 normal patients, including at least 3, at least 4, at least 5, at least 10, at least 20, and at least 50. In a further embodiment, the ranges and values of expression for a normal patient are derived using a statistical sampling of the population, or a statistical sampling of the area, ethnic group, age group, social group, or sex. In a further embodiment, the range and values of gene expression for a normal patient are derived from the patient before disease or during remission.

The results of the signature can be used in any one or more of the methods disclosed herein. Alternatively, one or more of the analyses can be included in one chip or array. The specific signature can include the results of the expression levels of one or more genes in that specific patient. In one embodiment, the signature is the results of the expression levels of at least 10 genes, preferably 40 genes, however, the signature can include the results of 50, 60, 70, 80, 90, 100, 150, 200, 250, 500, 750, 1000, 2000, 5000, and 10,000 genes which have been identified as being differentially expressed in RA. Some genes are more important or more involved in the manifestation or activation of the disease. Thus, the signature can require fewer genes when those that are more important have been identified and included.

In one embodiment, the results of the signature are used in a method of diagnosis. The method of diagnosis can include, a method of diagnosis of rheumatoid arthritis, a method of diagnosis of severity of the disease, a method of diagnosis of a manifestation of the disease and can include any or all of the above. Many of the same genes that are differentially expressed or involved in the manifestation of RA can also be involved in a different autoimmune disease. Alternatively, many of the same genes that are differentially expressed or involved in the manifestation of RA can also be involved in a different arthritide. Thus, the method of diagnosis can diagnose an arthritic or autoimmune disease, including, but not limited to, Lupus, Juvenile RA, Ankylosing Spondylitis, gout, osteoarthritis, fibrositis and fibromyalgia, Scleroderma, and even the autoimmune manifestations of Lyme disease and Streptococcus infection.

In a further embodiment, the results of the signature can be used in a method for prognosis of disease. The prognosis in various patients can vary tremendously. Some patients may progress very rapidly and may need a very aggressive treatment plan. Other patients may have a very mild version and may progress very slowly, requiring a more subtle treatment plan. This can be important when considering side effects, quality of life, and patient needs.

In a further embodiment, the results of the signature are used in a method of identification of the most efficacious treatment for that specific disease and for that specific patient. The treatment and the response to a drug can depend on which genes are being expressed. For example, in its most simple form, a patient with little IL-2 expression would not be best treated using a treatment that targets IL-2. However, the choice of a treatment method can involve a number of factors besides the gene expression of specific genes, including, the form of the disease, the severity of the disease, the manifestation of the disease, and the needs and wants of the patient. Many of these factors can be identified using one of the methods included herein.

In a further embodiment, the results are used to identify single nucleotide polymorphisms (SNPs), mutations, or Restriction Fragment Length Polymorphisms (RFLPs) associated with RA or other autoimmune diseases or other arthritides. The genes that are identified can be included in one or all of the genechips, arrays or analyses herein. In an alternative embodiment, a genechip that includes single nucleotide polymorphisms (SNPs), mutations, or Restriction Fragment Length Polymorphisms (RFLPs) is produced and used for diagnosis, prognosis, and/or identification of the best treatment or drug for use in treating RA.

Method of Identifying Targets for Drugs

In a further embodiment, the results of the signature are used to identify drug targets. Any or all of the genes identified herein and included in the signature or on a rheumatoid arthritis array can be used to further identify drugs or treatments that would target that gene or gene product.

Methods of identifying targets can include any method known to one of skill in the art, including, but not limited to: producing and testing small molecules, oligonucleotides (including antisense, RNAi and triplex formers), antibodies, and drugs that target any of the genes or gene products identified herein. Alternatively, gene therapy can be used to down-regulate, up-regulate, or express proteins or gene products identified herein.

The present methods will be further described by use of the following examples.

### EXAMPLES

In some of the following examples, the paws of mice with collagen-induced arthritis were analyzed in early disease and late disease by isolation of the RNA and microarray analysis. The results were confirmed using RT-PCR and in situ hybridization. Down- and up-regulation of genes was identified and the genes were clustered into groups. Human homologs are identified and the expression patterns are used to diagnose RA, to analyze the severity of disease in a patient, and to identify new treatments for arthritis. A number of genes were identified that previously had not been identified as being involved in arthritis; the genes thus identified can represent gene targets for drug therapy.

In the Examples relating to mouse experiments, DBA/1 mice were immunized with type II bovine collagen to induce arthritis, and mRNA was isolated from paws of non-immunized mice and from severely affected paws of mice at 28 days (acute disease model) and 49 days (chronic disease model) following the primary collagen injection. A single common reference control was used for all microarrays consisting of mRNA derived from the whole of a postnatal day 1 mouse, and all mRNAs were hybridized to duplicate microarrays (Incyte Pharmaceuticals, Inc., Palo Alto, Calif.). Among the 385 disease-specific genes differentially regulated in CIA are 102 expressed sequence tags (ESTs). Microarray analyses will help in further mapping out differences in gene expression between normal synovium and the synovium of acute and chronic CIA, including the identification of novel genes involved in arthritis.

### Example 1

### Production of Mice with Collagen-Induced Arthritis (CIA)

Mice with collagen-induced arthritis were used as a model for RA. Male DBA/IJ mice, 6 to 8 weeks of age, were purchased from The Jackson Laboratory (Bar Harbor, Me.). Mice were housed in the animal care facility at The Children's Hospital Research Foundation (Cincinnati, Ohio) under Institutional Animal Care and Use Committee approved conditions. Arthritis was induced with bovine type II collagen (CII, Elastin Products Co., Owensville, Mo.), as previously described (Thornton, et al. J. Immunol (2000) 165:1557-1563), the disclosure of which is hereby incorporated by reference in its entirety. Briefly, mice were injected intradermally with 100 μg of CII in complete Freund's adjuvant (CFA) at the base of the tail on day 0, and a similar booster was administered on day 21. Mice were evaluated for arthritis using an established macroscopic scoring system ranging from 0 to 4 (0=no detectable arthritis, 1=swelling and/or redness of paw or one digit, 2=two joints involved, 3=three or four joints involved and 4=severe arthritis of the entire paw and digits). At day 28 (early disease) and day 49 (late disease) following primary immunization, mice were sacrificed. Hind paws with an arthritic score of four were removed for mRNA analysis and in situ hybridizations (ISH). Paws from mice of the same age not treated with CII were used as normal controls.

### Example 2

### mRNA Expression Profiling of Early and Late CIA

Differential gene expression in paws of mice with CIA was analyzed in early (day 28) and late (day 49) arthritis and compared to that of paws from normal mice. These time points were chosen based on earlier studies that demonstrated their correlation with distinct histologic appearance and mRNA expression patterns by RPA.

RNA was isolated from paws that were quick frozen in liquid nitrogen and stored at −80° C. Frozen paws were minced with a scalpel and homogenized with a Polytron Tissue Tearor (Biospec Products, Bartlesville, Okla.) in appropriate volumes of RNA Stat-60 (Tel-Test, Friendswood, Tex.). Total RNA was extracted from the tissue homogenates according to the manufacturer's instructions. Pooled total RNA from normal (4 paws), early arthritic (3 paws) and late arthritic (4 paws) paws was used to isolate polyA+ RNA by the Oligotex mRNA isolation kit (Qiagen, Valencia, Calif.) according to the manufacturer's instructions. RNA concentrations were measured by fluorometry using the Ribogreen RNA Quantification Kit (Molecular Probes, Inc., Eugene, Oreg.).

DNA microarray analysis was performed as follows: mRNA of a whole 1 day old mouse was used for normalization of gene expression levels across all six microarray chips. Competitive hybridizations with Cy3 labeled whole 1 day old mouse mRNA versus Cy5 labeled normal paw mRNA, Cy5 labeled early paw mRNA or Cy5 labeled late paw mRNA were performed. Each sample (normal, early and late) was labeled and hybridized to two microarray chips. Hybridizations were performed on the mouse GEM1 array by Incyte Genomics (Palo Alto, Calif.).

Primary data were examined using Incyte Gemtools software and GeneSpring version 4.0.4 software (Silicon Genetics, Redwood City, Calif.). Defective cDNA spots (irregular geometry, scratched, or <40% area compared to average) or spot fluorescence hybridizations with signal to noise ratios less than 2.5:1 were eliminated from the data set. Data sets were subjected to normalization first within each microarray experiment such that the median of the Cy5 channel was balanced against the ratio of the Cy3 channel (k*(MedianCy3)=MedianCy5, where k is the ratio of the median intensities in each). Each microarray contained control genes present as non-mammalian single gene “spikes” or “complex targets”. The complex targets consisted of probe-sets that contain a pool of cellular genes expressed in most cell types. In addition, each experimental mRNA sample was augmented with incremental amounts of non-mammalian gene RNA (2×, 4×, 16×, etc) to permit assessment of the dynamic range attained within each microarray. Little variation was observed across the microarray series with respect to the 192 control genes (not shown), providing support for inter-array comparisons of temporally regulated genes. Genes were clustered according to their expression pattern by subjecting the log-transformed data (R=log₂Cy5/(kCy3), where R is the log of the expression ratio for each gene) to the hierarchical tree clustering algorithm as implemented in the GeneSpring program (Silicon Genetics). The hierarchical tree analysis was performed using a minimum distance value of 0.001, separation ratio of 0.5 and the standard correlation distance definition.

Mouse sense and antisense RNA probes were synthesized using the Stratagene RNA Transcription Kit (Stratagene, La Jolla, Calif.). T3 or T7 RNA polymerase produced ³⁵S-radiolabeled antisense or sense single-stranded RNA probes, respectively. A sense probe generated from an unrelated mouse gene was used as a negative control for in situ hybridization.

For early and late disease, mRNA from paws with severe arthritis (score of 4) were used to generate probes that were hybridized to Incyte Mouse GEM1 chips, as was mRNA from normal mouse paws. Hybridizations were conducted on duplicate chips, allowing for the elimination of genes whose expression levels differed by greater than 50% between the duplicate samples. 8,734 cDNAs, including known genes and ESTs, were represented on the microarray chip. 385 genes exhibited a greater than two-fold difference in expression between arthritic and normal paws and were selected for further analysis. Expression of 304 of these genes differed only between arthritic and normal paws, and expression of 81 of these genes differed between early and late arthritis. However, some of the genes identified were duplicates. Thus, the genes listed in Table 1 include some duplicates.

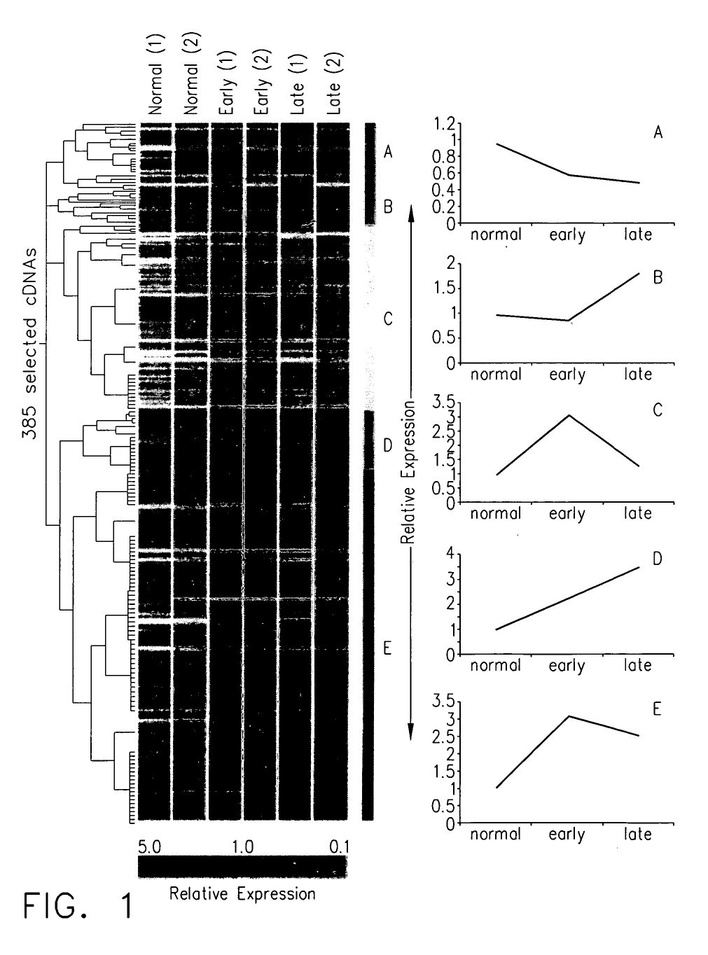

FIG. 1 demonstrates the 385 selected genes and their average levels of expression as compared to normal tissue values. The majority of genes were more highly expressed in arthritic paws as compared to normal paws. Genes were clustered according to their expression pattern during disease by hierarchical tree analysis. The resulting hierarchical tree structure revealed five distinct patterns of expression. Approximately half of the genes, represented by clusters D and E in Table 1 (225 genes, 58.4%), were upregulated both in early and late disease. It was possible to separate these genes into those with similar expression levels in early and late disease (cluster E in Table 1) and genes whose expression levels further increased during late disease (cluster D in Table 1). These may represent two distinct patterns or a continuum of coordinately regulated gene groups. Cluster C in Table 1 (105 genes, 27.3%) represents genes principally upregulated in early disease. Cluster B in Table 1 (18 genes, 4.7%) represents genes predominantly upregulated in late disease. Cluster A in Table 1 (37 genes, 9.6%) represents genes downregulated during both early and late disease, compared to normal paws. The individual genes and the number of ESTs belonging to each cluster are listed in Table 1. Please see Table 2 for the EST accession number and Table 3 for a schematic representation of the characteristics of Clusters A through E.

TABLE 1
Sequences - Human Homologs and Accession Numbers

| | | | | |
|---|---|---|---|---|
| | | Human | Human | |
| Mouse # | Name (mouse) | mRNA # | Protein # | Genbank Source |

Cluster A:

| | | | | |
|---|---|---|---|---|
| W09829 | trefoil factor 2 (spasmolytic protein 1) | NM_005423 | NP_005414 | AH003622 |
| W36838 | uteroglobin | NM_003357 | NP_003348 | BC004481 |
| AA028678 | palate, lung, and nasal epithelium | NM_016583 | NP_057667 | BC012549 |
| | expressed transcript | | | |
| AA047966 | four and a half LIM domains 1 | NM_001449 | NP_001440 | BC010998 |
| AA108401 | solute carrier family 27 (fatty acid | NM_003645 | NP_003636 | D88308 |
| | transporter) | | | |
| AA145089 | potassium voltage-gated channel, | NM_000238 | NP_000229 | U04270 |
| | subfamily H, member 2 | | | |
| AA241859 | betaine-homocysteine methyltransferase | NM_001713 | NP_001704 | U50929 |
| AA271284 | myoglobin | NM_005368 | NP_005359 | X00371, X00372, X00373 |
| AA261313 | nuclear receptor subfamily 1, group H, | NM_005123 | NP_005114 | U68233 |
| | member 4 | | | |
| AA275042 | amine N-sulfotransferase | NM_001054.1 | NP_001045 | 59% homologous |
| AA268120 | cytochrome P450, steroid inducible 3a11 | NM_007818 | NP_001045 | X 60452 |
| AA501052 | cardiac morphogenesis | | | 62% homologous |
| | | | | AW755250 |

Cluster B:

| | | | | |
|---|---|---|---|---|
| W11965 | enolase 3, β muscle | NM_001976 | NP_001967 | X51957, X56832 |
| W64550 | tumor-associated calcium signal | NM_002353 | NP_002344 | X77753 |
| | transducer 2 | | | |
| AA388939 | IG α chain C region | NM_001810 | NP_001801 | AL109804 |
| W34420 | ATPase, Ca++ transporting, cardiac | NM_004320 | NP_004311 | AH005190 |
| | muscle, fast twitch 1 | | | |
| AA015155 | S100 calcium binding protein A3 | NM_002960 | NP_002951 | Z18948 |
| AA204246 | Mus musculus dystonin (Bpag1-n) | NM_001723 | NP_001714 | L11690, M69225 |
| W62819 | neuronal protein 3.1 | NM_004772 | NP_004763 | U30521 |
| W64937 | angiopoietin related protein 2 | | | AF007150, AI467954, |
| | | | | AI 081 |

Cluster C:

| | | | | |
|---|---|---|---|---|
| W82159 | Fc receptor, IgG, low affinity III | NM_000570 | NP_000561 | X16863 |
| AI894016 | complement component 1, q | XM_031238 | XP_031238 | AK057792, BC009016 |
| | subcomponent, c | | | |
| AI324436 | Phospholipase A2 group VII | NM_005084 | NP_005075 | U20157 |
| AA116505 | CD53 antigen | NM_000560 | NP_000551 | AH011005, M37033 |
| AA177717 | Interleukin 1 receptor, type I | NM_000877 | NP_000868 | M27492 |
| AI322933 | Interleukin 4 receptor, α | NM_000418 | NP_000409 | X52425 |
| AA220007 | CD68 antigen | NM_001251 | NP_001242 | S57235 |
| AA289476 | Chemokine (C—C) receptor 2 | NM_000647 | NP_000638 | U03882 |
| AA289559 | Ecotropic viral integration site 2 | NM_014210 | NP_055025 | AH002689 |
| AI508758 | CD14 antigen | NM_000591 | NP_000582 | X13334 |
| AA286506 | Interleukin 4 receptor, α | NM_000418 | NP_000409 | X52425 |
| AA467489 | Integrin β 2 (Cd18) | NM_000211 | NP_000202 | M15395 |
| AA423373 | Glycoprotein 49 A | NM_024318 | NP_077294 | AF041262 |
| AA435060 | Leucocyte specific transcript 1 | | | LY117 |
| AA475774 | Cathepsin C | NM_001814 | NP_001805 | AU076460, X87212 |
| AA497620 | small proline-rich protein 2A | NM_005988 | NP_005979 | X53064 |
| W11889 | Hemochromatosis | NM_000410 | NP_000401 | U60319 |
| W13905 | Fibrinogen/angiopoietin-related protein | NM_016109 | NP_057193 | AF153606, AF202636 |
| W96914 | lysyl oxidase | NM_002317 | NP_002308 | AF039290, AF039291 |
| AA080231 | mannosidase 2, α B1 | NM_000528 | NP_000519 | AH006687 |
| AA152885 | small inducible cytokine subfamily B | NM_006419 | NP_006417 | 47% homologous |
| | (Cys-X-Cys) | | | AF044197 |
| AA170386 | colony stimulating factor 2 receptor, β 2, | No human | | |
| | low-affinity | genes | | |
| AA201097 | protein tyrosine phosphatase, receptor | NM_002838 | NP_002829 | Y00062, Y00638 |
| | type, C | | | |
| AA197349 | baculoviral LAP repeat-containing 2 | NM_001166 | NP_001157 | L49431, U37547 |
| AA239171 | elastin | NM_000501 | NP_000492 | AH007100 |
| AA268708 | Mus musculus hypoxia induced gene 2 | No human | | |
| | (Hig2) | genes | | |
| AA259959 | CD37 antigen | NM_001774 | NP_001765 | X14046 |
| AA260521 | uncoupling protein 2, mitochondrial | NM_003355 | NP_003346 | AF096289 |
| AA274104 | interleukin 2 receptor, γ chain | NM_000206 | NP_000197 | D11086 |
| AA387058 | apoptotic protease activating factor 1 | NM_013229 | NP_037361 | |
| AA547555 | CDC28 protein kinase 1 | NM_001826 | NP_001817 | X54941 |
| AA140523 | Rac GTPase-activating protein 1 | NM_013277 | NP_037409 | AL136794 |
| AA521764 | receptor (calcitonin) activity modifying | NM_005854 | NP_005845 | AJ001015 |
| | protein 2 | | | |
| AA051654 | metallothionein 1 | M 10942 | AAA5999587 | 85% homologous |
| AA265259 | oncostatin receptor | NM_003999 | NP_003990 | U60805 |
| AA178121 | cathepsin S | NM_004079 | NP_004070 | AL356292, BC002642, B |
| | | | | Q006623, M90696 |
| AA210306 | a disintegrin and metalloproteinase | NM_003816 | NP_003807 | U41766 |
| | domain 9 | | | |
| AA230451 | S100 calcium binding protein A8 | NM_002964 | NP_002955 | A12027, Y00278 |
| | (calgranulin A) | | | |
| AA268219 | macrophage expressed gene 1 | No human | | |
| | | genes | | |
| W41459 | Eukaryotic translation initiation factor | NM_001412 | NP_001403 | L18960 |
| | 1A | | | |
| AA003549 | Homolog of human ftp-3 | NM_003011 | NP_003002 | M93651 |
| AA063753 | ATP-binding cassette, sub-family A | NM_005502 | NP_005493 | AF165281, AF275948 |
| | (ABC1), member 1 | | | |
| AI594919 | Intersectin (SH3 domain protein 1 A) | NM_003024 | NP_003015 | AF064244 |
| AI385509 | Nuclear factor of κ light polypeptide | NM_002502 | NP_002493 | X61498 |
| | enhancer p49/p100 | | | |
| AA087193 | Lipocalin 2 | NM_005564 | NP_005555 | X99133 |
| AA175094 | Myristoylated alanine rich protein kinase | NM_002356 | NP_002347 | D10522 |
| | C substrate | | | |
| AI451276 | SH3 domain protein 3 | NM_012383 | NP_036515 | BC007459 |
| AA209640 | Histocompatibility 2, complement | NM_001710 | NP_001701 | BC004143, L15702 |
| | component factor B | | | |
| AA276440 | Selenoprotein P, plasma, 1 | NM_005410 | NP_005401 | Z11793 |
| AA432934 | Neuropilin | NM_003873 | NP_003864 | AF018956 |
| AA499926 | Peptidylprolyl isomerase A | NM_021130 | NP_066953 | X52851 |
| AA538499 | Phosphatidylinositol-4-phosphate | NM_005028 | NP_005019 | BC018034 |
| | 5-kinase, type II, α | | | |
| AA414612 | Capping protein α 1 | NM_006135 | NP_006126 | U56637 |
| W42321 | Pentaxin related gene | NM_002852 | NP_002843 | X63613 |
| AA162537 | Type II transmembrane protein MDL-1 | NM_013252 | NP_037384 | AJ271684 |
| AI646186 | Schlafen 4 | NM_018042 | NP_060512 | 41% homologous |
| AA462202 | BP-3 alloantigen | NM_004334 | NP_004325 | D21878 |
| AI322278 | Pyruvate dehydrogenase kinase 4 | NM_002612 | NP_002603 | U54617 |
| W98241 | proprotein convertase subtilisin/kexin | NM_006200 | NP_006191 | BC012064 |
| | type 5 | | | |
| AA172456 | small inducible cytokine A12 | NM_006273 | NP_006264 | X71087 |
| AA178155 | small inducible cytokine A4 | NM_002984 | NP_002975 | J04130 |
| AA267811 | lymphocyte cytosolic protein 2 | NM_005565 | NP_005556 | U20158 |
| AA144482 | chemokine (C—C) receptor 5 | NM_000579 | NP_000570 | AH005786 |
| AA266002 | B-cell leukemia/lymphoma 3 | NM_005178 | NP_005169 | M31732 |

Cluster D:

| | | | | |
|---|---|---|---|---|
| AA064293 | cartilage oligomeric matrix protein | NM_000095 | NP_000086 | L32137 |
| AI552105 | alkaline phosphatase 2, liver | NM_000478 | NP_000469 | AB011406, AH005272 |
| AA145458 | fibronectin 1 | NM_002026 | NP_002017 | M15801, X02761 |
| AA177218 | IG α chain C region | NM_001810 | NP_001801 | AL109804 |
| W63981 | fibromodulin | NM_002023 | NP_002014 | U05291, X75546 |
| AA030995 | peptidylprolyl isomerase B | NM_012117 | NP_036249 | S62077 |
| AA241281 | aquaporin 1 | NM_000385 | NP_000376 | M77829, U41517 |
| AA260949 | growth arrest and DNA-damage- | NM_006705 | NP_006696 | AF079806, AF265659 |
| | inducible, γ | | | |
| AA518165 | tissue inhibitor of metalloproteinase 2 | | A37128 | 100% homologous |
| W33786 | procollagen, type VI, α 1 | NM_001848 | NP_001839 | M20776, X15879, |
| AA000107 | procollagen, type XI, α 2 | NM_080679 | NP_542410 | AH006115, U32169 |
| AI323131 | thrombospondin 3 | NM_007112 | NP_009043 | L38969 |
| AA073904 | dickkopf homolog 3 (Xenopus laevis) | NM_013253 | NP_037385 | AF177396 |
| AA109900 | hemoglobin α, adult chain 1 | | P01922 | 85% homologous |
| AA537116 | immunoglobulin superfamily with | NM_005545 | NP_005536 | AB003184 |
| | leucine-rich repeats | | | |
| AI327504 | eukaryotic translation elongation factor 1 | NM_001958 | NP_001949 | X70940 |
| | α 2 | | | |

Cluster E:

| | | | | |
|---|---|---|---|---|
| W13151 | thymus cell antigen 1, τ | NM_006288 | NP_006279 | AL161958 |
| W54287 | Biglycan | NM_001711 | NP_001702 | AH002674, BC002416 |
| W89883 | procollagen, type III, α 1 | NM_000090 | NP_000081 | AI755052, M26939, X14420 |
| AA175226 | complement component 1, r | NM_001733 | NP_001724 | X04701 |
| | subcomponent | | | |
| AA242149 | FK506 binding protein 7 (23 kDa) | NM_017946 | NP_060416 | |
| AA209006 | complement component 1, s | | P09871 | 75% homologous |
| | subcomponent | | | |
| AA270625 | tenascin C | NM_015904 | NP_056988 | AF078035, AJ006776 |
| AA538511 | histocompatibility 2, L region | | S48134 | 69% homologous |
| W10072 | insulin-like growth factor 1 | NM_000618 | NP_000609 | X57025 |
| W14393 | Sid394p | NM_006815 | NP_006806 | BC 025957 |
| W11571 | hexokinase 1 | NM_022361 | NP_071756 | BC022323 |
| W14289 | cathepsin Z | NM_001336 | NP_001327 | AF136273 |
| W16254 | tubulin, β 5 | NM_001069 | NP_001060 | X79535 |
| W17813 | Talin | NM_006289 | NP_006280 | |
| W82677 | bone morphogenetic protein 1 | NM_001199 | NP_001190 | M22488 |
| W83904 | peptidylprolyl isomerase C | NM_000943 | NP_000934 | BC002678 |
| W89354 | procollagen-lysine, 2-oxoglutarate | NM_001084 | NP_001075 | BC011674 |
| | 5-dioxygenase 3 | | | |
| W99856 | procollagen, type V, α 1 | NM_000093 | NP_000084 | D90279, L38808, M76729 |
| AA002439 | annexin A5 | NM_001154 | NP_001145 | AH004914, J03745 |
| AA030294 | Mus musculus frizzled-1 | NM_003505 | NP_003496 | AB017363 |
| AA030780 | peroxisomal δ3, δ-2-enoyl-Coenzyme A | NM_006117 | NP_006108 | AF153612 |
| | isomerase | | | |
| AA038395 | Ras suppressor protein 1 | NM_012425 | NP_036557 | L12535 |
| AA060268 | phospholipase D3 | NM_012268 | NP_036400 | U60644 |
| AA067258 | Calumenin | NM_001219 | NP_001210 | AF013759, U67280 |
| AA110872 | amyloid β (A4) precursor protein | NM_000484 | NP_000475 | AH005295 |
| AA118715 | CD97 antigen | NM_001784 | NP_001775 | X84700 |
| AA122791 | histocompatibility 2, Q region locus 7 | | I37519 | 68% homologous |
| AA222201 | butyrate response factor 1 | NM_005141 | NP_005132 | J00129, M64983 |
| AA242611 | follistatin-like | NM_007085 | NP_009016 | BC000055 |
| AA397114 | annexin A4 | NM_001153 | NP_001144 | D78152, M82809 |
| AA259366 | Trp4-associated protein TAP1 | NM_015638 | NP_056453 | BC013144 |
| AA259551 | eukaryotic translation elongation factor 1 | NM_006452 | NP_006443 | BC010273 |
| | α 1 | | | |
| AA271275 | metallocarboxypeptidase CPX-1 | NM_019609 | NP_062555 | |
| AA260248 | growth factor receptor bound protein 10 | NM_005311 | NP_005302 | D86962 |
| AA437882 | ribosomal protein L9 | NM_000661 | NP_000652 | BG829769, U09953 |
| AA396298 | RNAse 4 | NM_002937 | NP_002928 | BC015520 |
| AA474964 | Lactotransferrin | NM_002343 | NP_002334 | X53961 |
| AA499296 | annexin A6 | NM_001155 | NP_001146 | J03578, X77673 |
| AA547428 | protein kinase, cAMP dependent, | NM_002731 | NP_002722 | M34181 |
| | catalytic, β | | | |
| W18828 | dihydropyrimidinase-like 3 | NM_001387 | NP_001378 | D78014 |
| AA048915 | guanine nucleotide binding protein, β-2, | NM_003922 | NP_003913 | U50078 |
| | related sequence 1 | | | |
| W14837 | protease, cysteine, 1 | NM_005606 | NP_005597 | BC003061 |
| W18376 | golgi vesicular membrane trafficking | NM_005868 | NP_005859 | AF007551 |
| | protein p18 | | | |
| W80177 | matrix metalloproteinase 2 | NM_004530 | NP_004521 | AH002654 |
| AA003452 | thrombospondin 4 | NM_003248 | NP_003239 | Z19585 |
| AA073604 | procollagen, type I, α 1 | NM_000088 | NP_000079 | Z74615 |
| AA124340 | transforming growth factor, β receptor | No human | | |
| | III | genes | | |
| AA241784 | insulin-like growth factor binding | NM_000599 | NP_000590 | |
| | protein 5 | | | |
| AA268082 | Lumican | NM_002345 | NP_002336 | BC007038 |
| AA260280 | procollagen, type III, α 1 | NM_000090 | NP_000081 | AI755052, M26939, X14420 |
| W13698 | FK506 binding protein 9 | NM_007270 | NP_009201 | BC011872 |
| W14113 | twist gene homolog, (Drosophila) | NM_000474 | NP_000465 | U80998, X99268 |
| AA052081 | Atpase, class I, type 8B, member 2 | No human | | |
| | | genes | | |
| W89518 | annexin A2 | MM_004039 | NP_004030 | D00017 |
| AI894006 | procollagen, type XI, α 1 | NM_001854 | NP_001845 | AU118365, J04177, U12139 |
| AA002481 | integrin β 5 | NM_002213 | NP_002204 | BC006541 |
| AA023549 | procollagen, type V, α 2 | NM_000393 | NP_000384 | BC015705, M58529, Y14690 |
| AA033050 | serine protease inhibitor 4 | NM_006216 | NP_006207 | BC015663 |
| AA037995 | microfibrillar associated protein 5 | NM_003480 | NP_003471 | AH007047 |
| AA059524 | procollagen, type VI, α 3 | NM_004369 | NP_004360 | X52022 |
| AA066921 | integral membrane protein 2 | NM_004867 | NP_004858 | AF038953 |
| AA108363 | ribosomal protein L3 | NM_000967 | NP_000958 | BC008492, BC012146 |
| AA108928 | secreted phosphoprotein 1 | NM_000582 | NP_000573 | AF052124 |
| AA220699 | transcobalamin 2 | NM_000355 | NP_000346 | AF047576, M60396 |
| AA272097 | fibroblast growth factor receptor 1 | NM_000604 | NP_000595 | M34641, X66945 |
| AA451495 | protocadherin 13 | NM_003735 | NP_003726 | |
| AA509765 | Endomucin | NM_016241 | NP_057325 | |
| AA542013 | fibroblast growth factor receptor 1 | NM_000604 | NP_000595 | M34641, X66945 |
| AA047991 | keratin complex 2, basic, gene 1 | NM_001004 | NP_000995 | BC005354, BC005920, BC007573 |
| W17771 | cathelin-like protein | NM_004345 | NP_004336 | Z38026 |
| AA221044 | histocompatibility 2, L region | | S48134 | 69% homologous |
| AI322868 | myristoylated alanine rich protein kinase | NM_002356 | NP_002347 | D10522 |
| | C substrate | | | |
| AA024088 | SH3 domain protein 3 | NM_012383 | NP_036515 | BC007459 |
| W18121 | histocompatibility 2, complement | NM_001710 | NP_001701 | BC004143, L15702 |
| | component factor B | | | |
| AA266975 | cell division cycle 42 homolog | NM_001791 | NP_001782 | AL121735, BC003682, M57298 |
| | (S. cerevisiae) | | | |
| AA172527 | ATP-binding cassette, sub-family G, | NM_004915 | NP_004906 | X91249 |
| | member 1 | | | |
| AA175651 | caspase 11 | NM_004347 | NP_004338 | U28015 |
| AA260476 | calpain 6 | NM_014289 | NP_055104 | AL031117 |
| W98807 | FXYD domain-containing ion transport | NM_014164 | NP_054883 | AA044211, AA296696, AF161462, |
| | regulator 5 | | | BG025158 |
| AA068750 | stromal cell derived factor 1 | NM_000609 | NP_000600 | U16752 |
| AA109951 | β-2 microglobulin | NM_004048 | NP_004039 | AB021288 |
| AA200339 | secretory leukocyte protease inhibitor | NM_003064 | NP_003055 | M74444, X04470 |
| AA245698 | regulator of G-protein signaling 5 | NM_003617 | NP_003608 | AB008109 |
| AA268592 | transforming growth factor, β induced, | NM_000358 | NP_000349 | M77349 |
| | 68 kDa | | | |
| AA272807 | histocompatibility 2, class II antigen A, | NM_002122 | NP_002113 | L34083, L46875, M20431 |
| | α | | | |
| W10023 | catenin β | NM_001904 | NP_001895 | X87838 |
| W12260 | surfeit gene 4 | NM_017503 | NP_059973 | BC014411, BM789997 |
| W14138 | kallikrein 3, plasma | No human | | |
| | | genes | | |
| W14540 | histocompatibility 2, K region | | P18462 | 68% homologous |
| W34612 | transglutaminase 2, C polypeptide | NM_004613 | NP_004604 | M55153 |
| W64075 | proline rich protein expressed in brain | NM_014764 | NP_055579 | D31767 |
| W81878 | osteoblast specific factor 2 | NM_006475 | NP_006466 | D13666 |
| W82141 | lysosomal membrane glycoprotein 1 | NM_005561 | NP_005552 | J04182 |
| W82946 | benzodiazepine receptor, peripheral | NM_000714 | NP_000705 | AH000829, M36035, U12421 |
| AA119072 | ceroid-lipofuscinosis, neuronal 2 | NM_000391 | NP_000382 | AF039704 |
| AA123008 | membrane bound C2 domain containing | NM_015292 | NP_056107 | BC004998 |
| | protein | | | |
| AA137942 | immunoglobulin J chain precursor | | P01591 | 77% homologous |
| AA172867 | purine-nucleoside phosphorylase | NM_000270 | NP_000261 | X00737 |
| AA178779 | interferon concensus sequence binding | NM_002163 | NP_002154 | M91196 |
| | protein | | | |
| AA185869 | β-1,4 N-actylgalactosaminyltransferase | NM_001478 | NP_001469 | L76079, M83651 |
| AA209884 | guanine nucleotide binding protein (G | NM_004125 | NP_004116 | BC015391 |
| | protein), γ 10 | | | |
| AA241132 | coatomer protein complex, subunit γ 1 | NM_016128 | NP_057212 | AF100756 |
| AA230649 | histocompatibility 2, class II, locus Dma | NM_006120 | NP_006111 | X62744 |
| AA260654 | TG interacting factor | NM_003244 | NP_003235 | X89750 |
| AA268148 | eukaryotic translation elongation factor | NM_007086 | NP_009017 | AJ006266 |
| | 1-β homolog | | | |
| AA396152 | CD44 antigen | NM_000610 | NP_000601 | AJ251595 |
| AA271576 | receptor (TNFRSF)-interacting serine- | NM_003804 | NP_003795 | U50062 |
| | threonine kinase 1 | | | |
| AA276030 | ATPase-like vacuolar proton channel | NM_001694 | NP_001685 | BC009290, BI548787 |
| AA414089 | heterogeneous nuclear ribonucleoprotein | NM_014740 | NP_055555 | D21853 |
| | D-like | | | |
| AA413831 | p100 co-activator | NM_014390 | NP_055205 | U22055 |
| AA060205 | butyrate response factor 1 | NM_005141 | NP_005132 | J00129, M64983 |
| AA200393 | CTP synthetase homolog | NM_019857 | NP_062831 | AK024070 |
| AA416325 | ATX1 (antioxidant protein 1) homolog 1 | NM_004045 | NP_004036 | U70660 |
| | (yeast) | | | |
| AA198703 | apoptotic protease activating factor 1 | NM_001160 | NP_001151 | AF013263 |
| AA457927 | polypeptide N- | NM_020474 | NP_065207 | U41514, Y10343 |
| | acetylgalactosaminyltransferase 1 | | | |

TABLE 2
ESTs

| | | | | |
|---|---|---|---|---|
| | | Genbank | | |
| Mouse gene name | Human mRNA | Source | Genbank | Description |

Cluster A

| no known gene | no homologene | | AA217294.1 | Public domain EST {IMAGE: 653016} |
|---|---|---|---|---|
| no known gene | no homologene | | W41083 | ESTs, Weakly similar to AF127035_1 |
| | | | | calcium-activated chloride channel |
| | | | | protein 2 [H. sapiens] |
| no known gene | no homologene | | AA137298 | ESTs |
| no known gene | no homologene | | AA268133 | ESTs |
| no known gene | no homologene | | AA027728.1 | Public domain EST {IMAGE: 463464} |
| no known gene | no homologene | | AA209551 | ESTs |
| no known gene | no homologene | | AA395994 | ESTs |
| tridadin | NM_006073 | U18985 | AA466026 | ESTs, Moderately similar to triadin |
| | | | | [H. sapiens] |
| no known gene | no homologene | | AA080287 | ESTs |
| extracellular link | NM_006691 | AF118101 | AA269330 | ESTs, Moderately similar to AF118108_1 |
| domain containing 1 | | | | lymphatic endothelium-specific hyaluronan |
| | | | | receptor LYVE-1 [H. sapiens] |
| no known gene | no homologene | | AI450674 | ESTs, Moderately similar to T20D3.3 |
| | | | | [C. elegans] |
| no known gene | no homologene | | AA290313 | ESTs |
| retinoblastoma- | NM_006101 | AF017790 | W99015 | ESTs, Moderately similar to |
| associated protein | | | | retinoblastoma-associated protein |
| HEC | | | | HEC [H. sapiens] |
| no known gene | no homologene | | AA288562.1 | Public domain EST {IMAGE: 749337} |
| no known gene | no homologene | | AI595209 | ESTs |
| RAP 1 GTPASE | NM_002885 | M64788 | AI509969 | ESTs, Highly similar to RAP1 GTPASE |
| activating protein 1 | | | | ACTIVATING PROTEIN 1 [Homo sapiens] |
| syaptotogmin 1 | MM_005639 | M55047 | W15872 | ESTs |
| cystaithionine gamma | NM_001902 | S52028 | AA245993 | ESTs, Highly similar to CYSTATHIONINE |
| lyase | | | | GAMMA-LYASE [Homo sapiens] |
| tocopherol alpha | NM_000370 | D49488 | AA277652 | ESTs |
| transfer protein | | | | |
| no known gene | no homologene | | AA061834 | ESTs |
| ectodermal neural | NM_003633 | AF059611 | AI608121 | ESTs, Weakly similar to open reading |
| cortex | | | | frame [M. musculus] |
| no known gene | no homologene | | AA145023 | ESTs |
| no known gene | no homologene | | AA414733 | ESTs |
| no known gene | no homologene | | AA259388 | ESTs |
| no known gene | NM_017779 | AK000361 | AA254513 | ESTs |

Cluster B

| no known gene | no homologene | | W09957 | ESTs, Moderately similar to unnamed |
|---|---|---|---|---|
| | | | | protein product [H. sapiens] |
| no known gene | no homologene | | W33467 | ESTs |
| myosin binding | NM_004533 | X73113 | AI385497 | ESTs, Moderately similar to C-PROTEIN, |
| protein C | | | | SKELETAL MUSCLE FAST-ISOFORM |
| | | | | [Gallus gallus] |
| Latenet transforming | NM_003573 | Y13622 | AA268327.1 | Public domain EST {IMAGE: 733726} |
| growth factor beta | | | | |
| binding protein 4 | | | | |
| aggrecan 1 | NM_001135 | M55172 | AA396306.1 | Public domain EST {IMAGE: 803275} |
| no known gene | no homologene | | AA066452 | ESTs, Weakly similar to A45910 ultra- |
| | | | | high-sulfur keratin - mouse [; |
| | | | | M. musculus] |
| human retinoic acid | NM_002888 | U27185 | AI464827 | ESTs, Weakly similar to TIG1_HUMAN |
| receptor responder | | | | RETINOIC ACID RECEPTOR RESPONDER |
| protein 1 | | | | PROTEIN 1 [H. sapiens] |
| no known gene | no homologene | | AA038095.1 | Public domain EST {IMAGE: 472860} |
| no known gene | no homologene | | AA038926 | ESTs |
| creatine kinase | NM_001825 | J05401 | AI322288 | ESTs, Highly similar to CREATINE KINASE, |
| | | | | SARCOMERIC MITOCHONDRIAL PRECURSOR |
| | | | | [Rattus norvegicus] |

Cluster C

| no known gene | NO HUMAN CDNA | | AA221886 | ESTs |
|---|---|---|---|---|
| TNFa induced | NO HUMAN CDNA | | AA272372 | ESTs, Weakly similar to match to ESTs |
| adipose-related | | | | AA316181 [H. sapiens] |
| protein | | | | |
| no known gene | NO HUMAN CDNA | | AA547022 | ESTs, Weakly similar to TIA1_MOUSE |
| | | | | NUCLEOLYSIN TIA-1 [M. musculus] |
| no known gene | MM_032947 | AF313413 | AA239554 | ESTs |
| no known gene | NM_015696 | AK027683 | W35981 | ESTs, Moderately similar to GLUTATHIONE |
| | | | | PEROXIDASE [Schistosoma mansoni] |
| no known gene | NM_030782 | BC025305 | AA020034 | ESTs, Weakly similar to cleft lip and |
| | | | | palate transmembrane protein 1 [H. |
| | | | | sapiens] |
| no known gene | NO HUMAN CDNA | | AI530458 | ESTs, Moderately similar to unnamed |
| | | | | protein product [H. sapiens] |
| no known gene | NM_015242 | AY049732 | AA268881 | ESTs, Highly similar to KIAA0782 |
| | | | | protein [H. sapiens] |
| no known gene | NO HUMAN CDNA | | AA030366 | ESTs |
| sorting nexin 10 | NM_013322 | BC031050, | AA260397 | ESTs, Weakly similar to SDP8 |
| | | BC147978 | | [M. musculus] |
| FLJ13433 | NM_022496 | AK023495 | AA184337 | ESTs, Weakly similar to ACTZ_HUMAN |
| | | | | ALPHA-CENTRACTIN [M. musculus] |
| no known gene | no homologene | | AA537509.1 | Public domain EST {IMAGE: 949810} |
| no known gene | no homologene | | AA388607 | ESTs |
| no known gene | no homologene | | W09604 | ESTs, Highly similar to large I antigen- |
| | | | | forming beta-1,6-N-acetylglucosaminyl- |
| | | | | transferase [M. musculus] |
| no known gene | no homologene | | W83671 | ESTs, Weakly similar to proteolipid |
| | | | | protein 2 [M. musculus] |
| major vault protein | NM_005115, | AJ238510, | AA200827 | ESTs, Moderately similar to I53908 |
| | NM_017458 | AJ23519, | | major vault protein - rat |
| | | X79882 | | [R. norvegicus] |
| no known gene | no homologene | | W08116 | ESTs, Moderately similar to WDNM1 |
| | | | | PROTEIN [Rattus norvegicus] |
| no known gene | no homologene | | AA204090 | ESTs, Weakly similar to AF201951_1 |
| | | | | high affinity immunoglobulin epsilon |
| | | | | receptor beta subunit [H. sapiens] |
| no known gene | no homologene | | AA098237 | ESTs |
| MDS006: X006 | NM_020233 | BC001294 | AA138584 | ESTs |
| protein | | | | |
| VMP1: likely | NM_030938 | AF14006 | AA516913 | ESTs, Weakly similar to CG1534 gene |
| ortholog of rat | | | | product [D. melanogaster] |
| vacuole membrane | | | | |
| protein 2 | | | | |
| Hepcidin | NM_021175 | AJ277280 | W12913 | ESTs, Moderately similar to HEPC_HUMAN |
| antimicrobial peptide | | | | ANTIMICROBIAL PEPTIDE HEPCIDIN |
| | | | | PRECURSOR [H. sapiens] |
| transmembrane 7 | NM_003272 | AF027826 | AA189999 | DNA segment, Chr 13, Abbott 1 expressed |
| superfamily member | | | | |
| 1 | | | | |
| no known gene | no homologene | | AA122848 | ESTs |
| mitogen-activated | NM_145342, | AB018276, | W82121 | ESTs, Weakly similar to scaffold |
| protein kinase kinase | NM_015093 | AL117407 | | attachment factor B [R. norvegicus] |
| kinase 7 interacting | | | | |
| protein 2 | | | | |
| no known gene | no homologene | | AA261222 | ESTs |
| glycine | NM_001482 | S68805 | AA185055 | ESTs, Highly similar to GATM_RAT GLYCINE |
| amidinotransferase | | | | AMIDINOTRANSFERASE PRECURSOR |
| | | | | [R. norvegicus] |
| phosphoinositide-3- | NM_014308 | AF128881 | AA290057 | ESTs |
| kinase | | | | |
| no known gene | no homologene | | AA210357 | ESTs |
| FLJ20401 | NM_017805 | AK000408 | AI391280 | ESTs, Highly similar to unnamed protein |
| | | | | product [H. sapiens] |
| no known gene | no homologene | | AA178549 | ESTs |
| no known gene | no homologene | | W11587 | ESTs, Moderately similar to SARL_HUMAN |
| | | | | SARCOLIPIN [H. sapiens] |
| no known gene | no homologene | | AA163875 | ESTs |
| no known gene | no homologene | | AI595493 | ESTs, Weakly similar to AF161080_1 |
| | | | | inhibitory receptor PILRalpha |
| | | | | [H. sapiens] |
| KIAA0475 | NM_014864 | AB007944 | AA210038 | ESTs |
| no known gene | no homologene | | AA268055 | ESTs |
| FLJ22833 | NM_022837 | AK026486 | AA175979 | ESTs, Weakly similar to CG5181 gene |
| | | | | product [D. melanogaster] |
| placenta-specific 8 | NM_016619 | AF208846 | AA245029 | DNA segment, Chr 5, Wayne State |
| | | | | University 111, expressed |
| Z39IG: Ig | NM_007268 | AJ32502 | AA261076.1 | Public domain EST {IMAGE: 720457} |
| superfamily protein | | | | |

Cluster D

| no known gene | MM_05133 | AE006639 | W14214 | ESTs |
|---|---|---|---|---|
| procollagen, type V, | NM_000393 | BC015705, | AA138290 | ESTs |
| alpha 2 | | M58529, | | |
| | | Y14690 | | |
| solute carrier family | NM_004955 | U81375 | AI451844 | ESTs, Highly similar to AF131212_1 |
| 29, member 1 | | | | equilibrative nitrobenzylthioinosine- |
| | | | | sensitive nucleoside transporter ENT1 |
| | | | | [M. musculus] |
| no known gene | NO HUMAN CDNA | | W99891 | ESTs |
| solute carrier family | NM_004955 | U81375 | AA397253 | ESTs, Highly similar to AF131212_1 |
| 29, member 1 | | | | equilibrative nitrobenzylthioinosine- |
| | | | | sensitive nucleoside transporter ENT1 |
| | | | | [M. musculus] |
| no known gene | NO HUMAN CDNA | | W29300.1 | Public domain EST {IMAGE: 337567} |
| no known gene | NM_001908 | AK092070 | W41810 | ESTs, Weakly similar to T17344 |
| | | | | hypothetical protein DKFZp586L2024.1 - |
| | | | | human [H. sapiens] |
| twisted gastulation | NM_020648 | BC020490 | AA267373 | ESTs |
| protein | | | | |
| adenosine deaminase | NM_001112 | U76420 | W16053 | ESTs |
| RNA specific B1 | | | | |
| no known gene | NM_015429 | AB056106 | AA267567 | ESTs |
| oxoglutarate | NM_002541 | D10523 | W13320 | ESTs, Highly similar to 2-OXOGLUTARATE |
| dehydrogenase | | | | DEHYDROGENASE E1 COMPONENT PRECURSOR |
| | | | | [Homo sapiens] |
| no known gene | NM_016308 | AF070416 | AI594925 | ESTs, Highly similar to URIDYLATE KINASE |
| | | | | [Saccharomyces cerevisiae] |
| Mrps18b | NM_041046 | AF100761 | AI426268 | ESTs, Moderately similar to PTD017 |
| | | | | [H. sapiens] |

| | | | |
|---|---|---|---|
| no known gene | NO HUMAN CDNA | AA185432 | ESTs |
| no known gene | NO HUMAN CDNA | AA461746 | ESTs |

Cluster E

| no known gene | AA146022, | | AA146022 | ESTs |
|---|---|---|---|---|
| | AK026169 | | | |
| no known gene | NM_003505 | AB017363 | AI604159 | ESTs |
| no known gene | NO HUMAN CDNA | | W14925 | ESTs, Moderately similar to KIAA1029 |
| | | | | protein [H. sapiens] |
| no known gene | NM_014864 | AB007944 | AA274981 | ESTs |
| no known gene | NO HUMAN CDNA | | AA033308 | ESTs |
| N4wbp-4 pending | NM_020182 | AF305616 | AA144094 | ESTs, Highly similar to dJ718J7.1 |
| | | | | [H. sapiens] |
| filamin like protein | AI678681 | | AA466198 | ESTs, Highly similar to ENDOTHELIAL |
| | | | | ACTIN-BINDING PROTEIN [Homo sapiens] |
| no known gene | NM_004518 | Y15065 | W11395 | ESTs |
| secreted modular | NM_022138 | AB014737 | AA272826 | ESTs, Weakly similar to AF070470_1 |
| binding protein 2 | | | | SPARC-related protein [M. musculus] |
| no known gene | NM_007080 | AJ238098 | W09867 | ESTs, Moderately similar to HYPOTHETICAL |
| | | | | 9.3 KD PROTEIN ZK652.1 IN CHROMOSOME III |
| | | | | [Caenorhabditis elegans] |
| no known gene | NO HUMAN CDNA | | AA274099 | ESTs, Weakly similar to ZEP-kinase |
| | | | | [M. musculus] |
| no known gene | NM_017510 | BC001123 | AA517431 | ESTs, Moderately similar to GLYCOPROTEIN |
| | | | | 25L PRECURSOR [Canis familiaris] |
| no known gene | AL832340, | | AA386758 | ESTs |
| | AL833405 | | | |
| no known gene | NO HUMAN CDNA | | AA217009 | ESTs |
| transforming growth | NM_006022 | AJ222700 | AA060863.1 | Public domain EST {IMAGE: 482995} |
| factor beta 1 induced | | | | |
| transcript 4 | | | | |
| no known gene | NM_004265 | AF084559 | AA068575 | ESTs, Weakly similar to delta-6 fatty |
| | | | | acid desaturase [M. musculus] |
| cathepsin Z | NM_001336 | AF136273 | W14289 | DNA segment, Chr 2, Wayne State University |
| | | | | 143, expressed |
| janus kinase 1 | NM_002227 | M64174 | W29699 | ESTs, Highly similar to TYROSINE-PROTEIN |
| | | | | KINASE JAK1 [Homo sapiens] |
| no known gene | NO HUMAN CDNA | | W82178 | DNA segment, Chr 7, Wayne State University |
| | | | | 86, expressed |
| no known gene | NM_032849 | AK055635 | AA024250 | ESTs |
| no known gene | NO HUMAN CDNA | | AA002801.1 | Public domain EST {IMAGE: 426240} |
| no known gene | NM_001478 | L76079, | AA268669 | ESTs, Weakly similar to AF232669_1 |
| | | M83651 | | Kalirin-12a [R. norvegicus] |
| ATP binding cassette, | NM_005502 | AF165281, | AA203809 | ESTs |
| subfamily A (ABC1), | | AF275948 | | |
| member 1 | | | | |
| no known gene | NO HUMAN CDNA | | W36470 | ESTs, Weakly similar to T00343 |
| | | | | hypothetical protein KIAA0584 - |
| | | | | human [H. sapiens] |
| no known gene | NO HUMAN CDNA | | AA050516 | DNA segment, Chr 9, Wayne State University |
| | | | | 18, expressed |
| no known gene | NO HUMAN CDNA | | AI426270 | ESTs, Weakly similar to nuclear protein |
| | | | | np95 [M. musculus] |
| no known gene | NM_002358 | U31278 | AA466530 | ESTs, Moderately similar to KIAA0280 |
| | | | | [H. sapiens] |
| no known gene | NM_022763 | AK027052 | AA265864 | ESTs |
| golgi SNAP receptor | NM_004287, | AF007548, | AA002301 | ESTs |
| complex member 2 | NM_054022 | AF229796 | | |
| no known gene | NM_003387 | AF031588, | AA174503 | ESTs |
| | | AF106062 | | |
| lectin, mannose- | NM_005570 | U09716, | AA036111 | ESTs, Highly similar to ERGIC-53 PROTEIN |
| binding, 1 | | X71661 | | PRECURSOR [Homo sapiens] |
| ribosome binding | NM_004587 | AF006751 | AA002385 | ESTs, Moderately similar to KIAA1398 |
| protein 1 | | | | protein [H. sapiens] |
| no known gene | NO HUMAN CDNA | | AA139063 | ESTs, Moderately similar to KIAA0007 |
| | | | | [H. sapiens] |
| no known gene | NO HUMAN CDNA | | AA189695 | ESTs, Highly similar to TROPOMYOSIN 4, |
| | | | | EMBRYONIC FIBROBLAST ISOFORM |
| | | | | [Rattus norvegicus] |
| no known gene | NO HUMAN CDNA | | AI449320 | ESTs |
| quiescin Q6 | NM_002826 | U97276 | AA024091 | ESTs, Moderately similar to quiescin |
| | | | | [H. sapiens] |
| no known gene | NM_019026 | AB020980 | AA472933 | ESTs, Highly similar to unknown |
| | | | | [H. sapiens] |
| no known gene | NO HUMAN CDNA | | AA048837 | ESTs, Highly similar to INTERFERON- |
| | | | | INDUCIBLE PROTEIN [Rattus norvegicus] |
| no known gene | NO HUMAN CDNA | | AA239252 | ESTs |
| golgi reassembly | NO HUMAN CDNA | | AA213185 | ESTs, Weakly similar to AF218940_1 |
| stacking protein 2 | | | | formin-2 [M. musculus] |
| no known gene | NM_020123 | AF160213 | AA144167 | ESTs, Highly similar to unnamed protein |
| | | | | product [H. sapiens] |
| neuropilin 2 | NM_003872 | AF022860 | AA269699 | ESTs |
| no known gene | NO HUMAN CDNA | | AA217196 | ESTs |
| no known gene | NO HUMAN CDNA | | AA432472.1 | Public domain EST {IMAGE: 833346} |
| calreticulin | NO HUMAN CDNA | | W33774.1 | Public domain EST {IMAGE: 352406} |
| no known gene | NO HUMAN CDNA | | AA177584.1 | Public domain EST {IMAGE: 621742} |
| no known gene | NM_020820 | AJ320261 | W82294 | ESTs |
| no known gene | NM_018446 | AF157318 | AA210344 | ESTs, Highly similar to AF157318_1 |
| | | | | AD-017 protein [H. sapiens] |
| lectin, mannose- | NM_005570 | U09716, | AA244713 | ESTs, Highly similar to ERGIC-53 PROTEIN |
| binding, 1 | | X71661 | | PRECURSOR [Homo sapiens] |
| no known gene | NO HUMAN CDNA | | AA437983 | ESTs, Weakly similar to AF151373_1 |
| | | | | nucleolin-related protein NRP |
| | | | | [R. norvegicus] |
| no known gene | NM_001643 | BC007309 | AA404092 | ESTs, Moderately similar to COATOMER |
| | | | | DELTA SUBUNIT [Homo sapiens] |
| tripartite motif | NM_030912 | AF281046 | AA027381 | ESTs, Weakly similar to I49642 estrogen- |
| protein 8 | | | | responsive finger protein - mouse |
| | | | | [M. musculus] |
| no known gene | NM_014604 | AF028823 | AI893697 | ESTs, Highly similar to HYPOTHETICAL |
| | | | | 13.5 KD PROTEIN C45G9.7 IN CHROMOSOME |
| | | | | III [Caenorhabditis elegans] |
| no known gene | NO HUMAN CDNA | | AA265636 | ESTs, Highly similar to CALDESMON, |
| | | | | SMOOTH MUSCLE [Gallus gallus] |
| no known gene | NO HUMAN CDNA | | AA536838 | ESTs |
| filamin like protein | AI678681 | | AA003323 | ESTs, Highly similar to ENDOTHELIAL |
| | | | | ACTIN-BINDING PROTEIN [Homo sapiens] |
| no known gene | NM_020790 | AF201945 | W14353 | ESTs, Weakly similar to trabecular |
| | | | | meshwork-induced glucocorticoid |
| | | | | response protein [M. musculus] |
| no known gene | NO HUMAN CDNA | | AA260155 | DNA segment, Chr 2, Wayne State University |
| | | | | 127, expressed |
| platelet-derived | NM_006207 | D37965 | AA030377 | ESTs, Highly similar to PDGF receptor |
| growth factor | | | | beta-like tumor suppressor |
| receptor-like | | | | [H. sapiens] |
| no known gene | NM_014933 | AB018358 | AA544844 | ESTs, Moderately similar to T14150 |
| | | | | vesicle associated protein 1 - rat |
| | | | | [R. norvegicus] |
| no known gene | NO HUMAN CDNA | | W10776.1 | Public domain EST {IMAGE: 314509} |
| no known gene | NM_032849 | AK055635 | AI552496 | ESTs |
| no known gene | NM_004394 | X76105 | AA269524 | ESTs, Highly similar to DAP1_HUMAN |
| | | | | DEATH-ASSOCIATED PROTEIN 1 |
| | | | | [H. sapiens] |
| no known gene | NO HUMAN CDNA | | W97172 | DNA segment, Chr 13, Wayne State University |
| | | | | 115, expressed |
| no known gene | NM_012426 | D87686 | AA269584 | ESTs, Highly similar to KIAA0017 protein |
| | | | | [H. sapiens] |
| enolase 1, alpha non | NM_001428 | X16287 | AA204262 | ESTs, Highly similar to ALPHA ENOLASE |
| neuron | | | | [Mus musculus] |

| | | | |
|---|---|---|---|
| no known gene | NO HUMAN CDNA | AA172597 | ESTs |
| no known gene | NO HUMAN CDNA | AA237920 | ESTs |

TABLE 3
Characteristics of Clusters A through E

| Cluster | Early | Late |
|---|---|---|
| A | ↓ | ↓ |
| B | — | ↑ |
| C | ↑ | — |
| D | ↑ | ↑↑ |
| E | ↑ | ↑ |

↓ = gene expressed reduced at least 2 fold.
↑ = gene expression increased at least 2 fold.
↑↑ = gene expression increased more than 2 fold.

### Example 3

### Confirmation of Microarray Data by RT-PCR and In Situ Hybridization

Confirmation of the microarray data was performed by measuring the expression level of genes in two individual paws at each time point using real time RT-PCR and in situ hybridization.

Real time reverse transcription (RT) PCR analysis was performed as follows: to remove possible genomic DNA contamination, total paw RNA was treated with amplification grade DNAse I (Gibco Life Technologies, Rockville, Md.). RNA was then subjected to reverse transcription using SUPERSCRIPT Preamplification System for First Strand cDNA Synthesis (Gibco Life Technologies). Serial dilutions of the cDNA template were prepared and PCR was carried out using a Lightcycler System (Roche Molecular Biochemicals, Palo Alto, Calif.). After each elongation phase, the fluorescence of SYBR Green I, which binds double-stranded DNA was measured. Reactions (20 μl) were performed in microcapillary tubes using 5 μl of diluted cDNA with SYBR Green I (Roche Molecular Biochemicals), master mix, upstream and downstream primers and MgCl₂. Sequences of primer pairs were as follows: Follistatin-like, upstream: 5′-GGA TTG AGA ATC AGC ACT GGG-3′ (SEQ ID NO:386); downstream: 5′-TTG AAA GGG AGG GCA CAG AAC-3′ (SEQ ID NO:387); IL-2Rα, upstream: 5′-CGG AAG CCT GAA CAT CAA TCC-3′ (SEQ ID NO:388); downstream: 5′-GCC ACT AAC CCC AAC TCT TAT GAG-3′ (SEQ ID NO:389); GAPDH, upstream: 5′-ACC ACA GTC CAT GCC ATC AC-3′ (SEQ ID NO:390); downstream: 5′-TCC ACC ACC CTG TTG CTG TA-3′ (SEQ ID NO:391). Reactions containing water or cDNA synthesized without reverse transcriptase, as template, resulted in no PCR products. Dilutions of cDNA synthesized from early paw RNA were predicted to have the highest expression of the gene product being amplified and, thus, were used as the concentration standards. Lightcycler quantification software v3 was used to compare amplification in experimental samples during the log-linear phase to the standard curve from the dilution series of acute tissue. All experimental samples were normalized to GAPDH (glyceraldehyde-3-phosphate dehydrogenase) expression levels for that tissue. Expression levels of each gene were plotted relative to the levels in normal tissue.

In situ hybridization analysis was performed as previously described (Witte, et al. Am J. Pathol 1991;139:717-724). Briefly, ten micron cryostat sections of snap frozen tissue were air dried on TESPA coated Superfrost Plus (Histology Control Systems, Glenhead, N.Y.) slides and post-fixed in 4% (w/v) paraformaldehyde in PBS then acetylated with acetic anhydride as described. Paws were fixed for 48 hours in 4% (w/v) paraformaldehyde (Electron Microscopy Sciences, Ft. Washington, Pa.) in PBS at 4° C. immediately after harvesting. Following fixation, the tissue was decalcified in TBD-2 (Shandon, Pittsburgh, Pa.). Complete decalcification of the tissue was determined using 5% ammonium oxalate. Following decalcification the tissue was rinsed for ten minutes in running water and placed in 30% sucrose in PBS for 24 hours at 4° C. The samples were embedded in M-1 mounting media (Shandon), frozen in liquid nitrogen and stored at −80° C. Hybridizations were done overnight at 45° C. under a sealed coverslip. Following hybridization, the sections were treated with RNAse to remove unbound probe and the slides were washed extensively under highly stringent conditions. The slides were developed in Kodak D19 developer (Rochester, N.Y.). Sections were counterstained with hematoxylin & eosin and photographed using both dark- and bright-field illumination.

Mouse sense and antisense RNA probes were synthesized using the RNA Transcription Kit (Stratagene, La Jolla, Calif.). T3 or T7 RNA polymerase produced ³⁵S-radiolabeled antisense or sense single-stranded RNA probes, respectively. A sense probe generated from an unrelated mouse gene was used as a negative control for in situ hybridization.

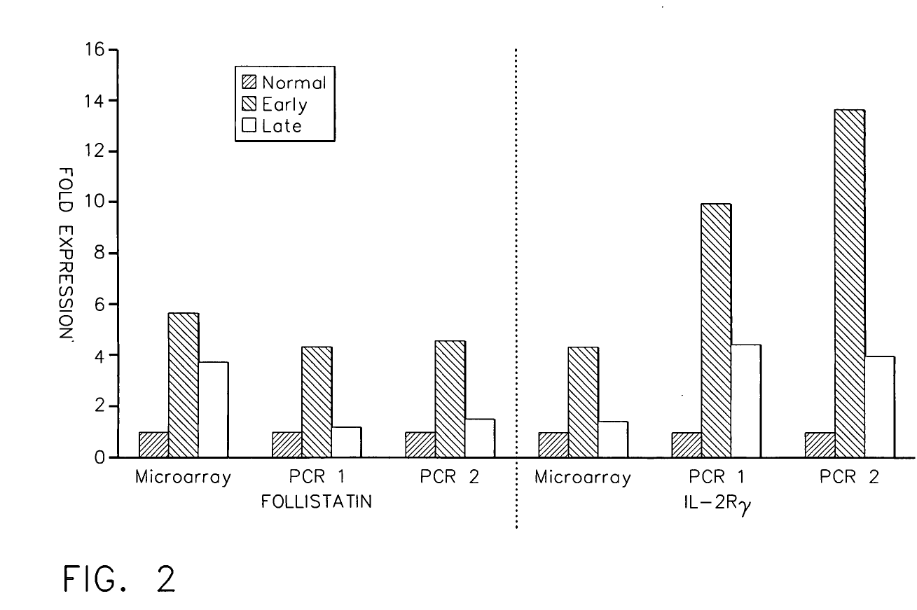

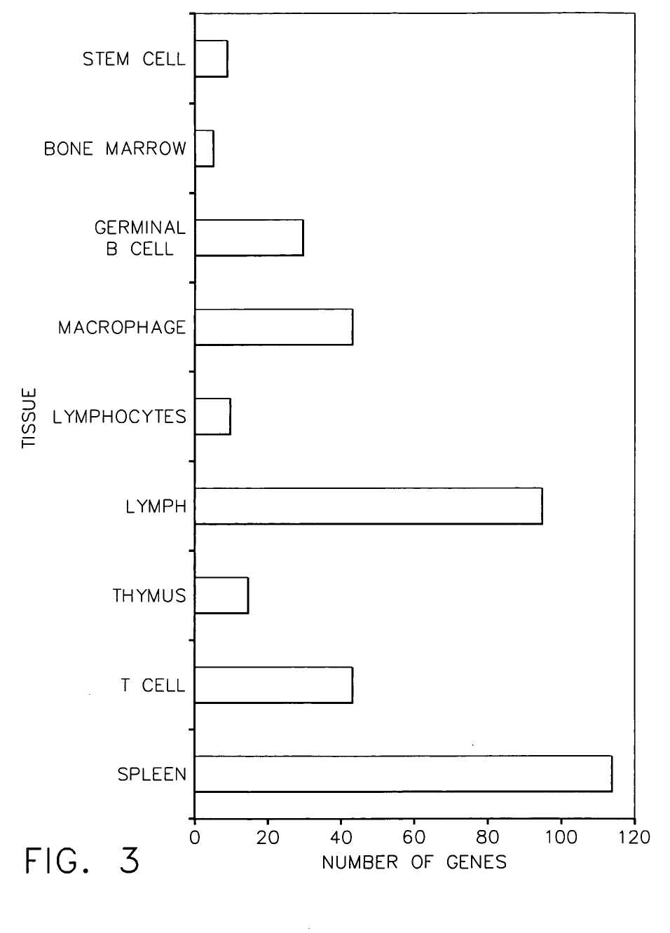

Although none of the genes previously demonstrated to be upregulated by RPA were present on the microarray chip, two genes on the DNA microarray were related to genes whose expression patterns we have previously analyzed by RPA. One of the genes, IL-2Rγ, had a similar expression pattern to the previously observed expression pattern of IL-2. Another gene, follistatin-like, which is induced by TGFβ, had a similar expression pattern to the previously observed expression patterns of TGFβ 1, 2 and 3. Comparison of the expression of follistatin-like and IL-2Rγ by microarray and real time RT-PCR revealed similar patterns of expression (FIG. 2). In addition, spatial expression of IL-2Rγ was analyzed by in situ hybridization (FIG. 3). The expression pattern matched that observed in the DNA microarray hybridizations. IL-2Rγ was expressed in the inflammatory tissue surrounding the joint and in the periosteal tissue along the length of the bone.

### Example 4

### Classification of Differentially Expressed Genes

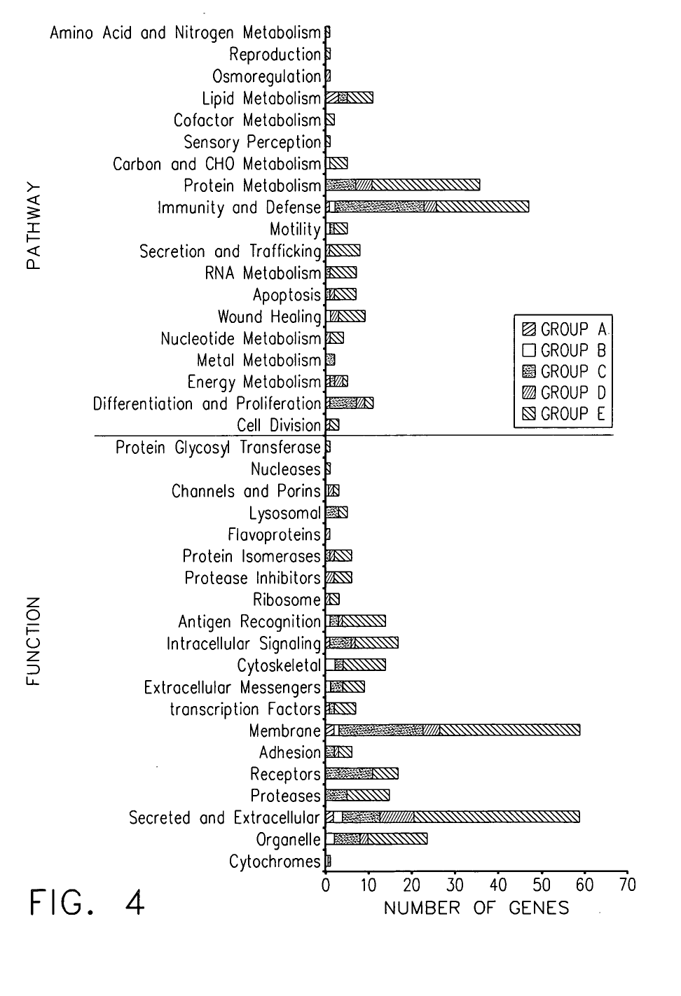

Of the 385 genes that were found to be differentially expressed during CIA in the mouse paw, 102 were expressed sequence tags (ESTs) and preferred members of this group represent novel genes critical to the pathology of CIA. Excluding duplicate gene spotting on the chip, 240 of the 385 gene sequences are annotated genes. Information on their expression in various tissues was obtained using LocusLink and Unigene at the National Center for Biotechnology Information (NCBI) website (ncbi.nlm.nih.gov/). These genes have been reported in a variety of tissues, including but not limited to bone, brain, colon, liver, lung, kidney, mammary, skin, spleen and testis. Not surprisingly, the majority are expressed in the lymphoid organs, including spleen and lymph nodes (FIG. 4).

To further characterize the annotated genes, they were grouped into categories using Incyte's Function and Pathways categorization (FIG. 5). The largest functional categories included immunity and defense (47 genes), protein metabolism (36 genes), lipid metabolism (11 genes) and differentiation and proliferation (11 genes). The largest pathways categories included membrane (59 genes), secreted and extracellular (59 genes), organelle (24 genes), intracellular signaling (17 genes), receptors (17 genes), proteases (15 genes) and antigen recognition (14 genes). In most cases, the genes in each category were distributed proportionally to the size of the clusters identified in FIG. 1.

The 240 previously characterized genes that were differentially regulated during CIA were analyzed through extensive literature searches. Of these 240 genes, a number of genes that have not previously been characterized in autoimmune arthritis but that could potentially be involved, were identified. From the literature searches on these particular genes, a number of genes were found to be associated with three basic biological functions. These genes, as well as their temporal expression, are listed in Table 4.

TABLE 4
Genes novel to arthritis

| | |
|---|---|
| Early | Late |

Proliferation, differentiation and tumorigenesis

| | | |
|---|---|---|
| enolase 3, β muscle | | * |
| tumor-associated calcium signal transducer 2 | | * |
| S100 calcium binding protein A3 | | * |
| angiopoietin related protein 2 | | * |
| β-1,4 N acetylgalactosaminyltransferase | * | * |
| polypeptide N-acetylgalactosaminyltransferase 1 | * | * |
| endomucin | * | * |
| growth factor receptor bound protein 10 | * | * |
| growth arrest and DNA-damage-inducible, γ | * | * |
| dickkopf homolog 3 (Xenopus laevis) | * | * |
| CDC28 protein kinase 1 | * | |
| a disintegrin and metalloproteinase domain 9 | * | |
| ecotropic viral integration site 2 | * | |
| selenoprotein P | * | |
| proprotein convertase subtilisin/kexin type 5 | * | |
| B-cell leukemia/lymphoma 3 | * | |

Apoptosis

| | | |
|---|---|---|
| apoptotic protease activating factor 1 | * | * |
| regulator of G-protein signaling 5 | * | * |
| calumenin | * | * |
| CD97 | * | * |
| calpain 6 | * | * |
| caspase 11 | * | * |
| receptor interacting protein | * | * |
| transglutaminase 2, C polypeptide | * | * |
| CD44 | * | * |
| CD53 | * | |
| fibrinogen/angiopoietin-related protein | * | |
| baculoviral IAP repeat-containing 2 | * | |
| uncoupling protein 2, mitochondrial | * | |

Inflammation

| | | |
|---|---|---|
| annexin A2 | * | * |
| annexin A4 | * | * |
| annexin A6 | * | * |
| lysosomal membrane glycoprotein 1 | * | * |
| protocadherin 13 | * | * |
| catenin beta | * | * |
| pentaxin related gene | * | |
| small proline-rich protein 2A | * | |
| small inducible cytokine subfamily B (Cys-X-Cys) | * | |
| colony stimulating factor 2 receptor, | * | |
| β 2, low-affinity | | |
| CD37 | * | |
| type II transmembrane protein | * | |
| BP-3 alloantigen | * | |
| Mus musculus hypoxia induced gene 2 (Hig2) | * | |

The present study quantitatively analyzed coordinated gene expression on a global scale from paws of mice with CIA to identify novel genes involved in arthritis as well as to identify gene expression patterns that differ between early and late synovitis in this model system. Genes known to be upregulated in CIA or RA were confirmed by the analysis. However, most of the differentially-expressed genes identified by the microarray have not been previously described in arthritis.

The difference in expression profiles observed between early and late disease has not previously been fully-appreciated. Even though the microarray analysis was limited to two time points over the course of the disease, cluster analysis grouped the 385 genes according to their mRNA expression in early versus late disease. In some embodiments, the hierarchical clusters can represent coordinately expressed genes, the effects of cell phenotype and/or a combination of the two. Confirmation of the validity of the microarray expression analysis includes RT-PCR analysis of expression of follistatin-like gene and IL-2Rγ, as well as analysis of the spatial expression of IL-2Rγ by in situ hybridization. Of 385 genes on the microarray found to be differentially expressed in CIA, 240 have been previously annotated. These 240 genes can be divided into several biological functions and pathways; however, none of the clusters were over-represented in any of these categories.

Included in the group of annotated genes are many that have previously been demonstrated to be upregulated in RA, including TIMP-3, β-2 microglobulin, biglycan, lumican, insulin-like growth factor binding protein 5 and stromal cell derived factor-1, as well as proinflammatory genes such as IL-2Rγ, small inducible cytokine A12 and A4 (MCP5 and MIP1β respectively), CCR5, macrophage expressed gene 1, cathepsins C and S, CD14 and fibronectin. Expression of a majority of these 240 genes also occurs in lymphoid organs, which is expected since the synovial inflammation is dominated by immune cells.

The 240 annotated genes were analyzed through extensive review of the literature, resulting in a list of 43 genes not previously characterized in autoimmune arthritis. Based on their known biological functions these genes might play central roles in the pathophysiology of the disease. These genes, as well as their temporal expression, are listed in Table 4. Several interesting comparisons can be made between the biological function of these genes, their temporal expression patterns, and the histopathologic appearance of arthritis.

### Example 5

### Genes Expressed Throughout CIA

Several genes involved in cell proliferation, differentiation and tumorigenesis were upregulated throughout the disease (clusters D and E). These included β-1,4 N-acetylgalactosaminyltransferase and polypeptide N-acetylgalactosaminyltransferase 1, that are involved in the synthesis of gangliosides, whose overexpression is associated with a marked increase in growth rate and invasive activity.

Numerous genes involved in apoptosis were identified that were expressed both in early and late disease. Cellular turnover in normal tissues is tightly regulated through a balance of cell proliferation and cell death. The regulation of cell populations within the joint is very likely also controlled by apoptotic processes. Apoptosis of cells within the arthritic joint has been proposed to be a source of self-peptides that could generate auto-antigens that may propagate inflammation. One of these, CD44, has been postulated to play a role in the elimination of neutrophils from sites of inflammation in inflammatory kidney disease and its upregulation on the surface of chondrocytes may contribute to cartilage degeneration in RA patients. Other genes include calpain 6 and caspase 11, which are members of two families of cysteine proteases involved in the regulation of pathological cell death. Additionally, receptor interacting protein (RIP) interacts with Fas, causing morphological changes in cells that resemble apoptosis.

Inflammatory processes occur both early and late in disease. Therefore, the identification of genes involved with inflammation was not unexpected; however, various genes were identified that had not previously been associated with inflammation in CIA or RA. These genes include annexins A2, A4 and A6, which affect the activation and migration of macrophages. The human homologue of lysosomal membrane glycoprotein 1, h-LAMP1, is detectable in patients with scleroderma and systemic lupus erythematosus and may contribute to the migration of activated leukocytes to the sites of inflammation. Catenin-β, when complexed with E-cadherin, is upregulated in gut inflammation of patients with spondyloarthropathy.

### Example 6

### Genes Expressed in Late CIA

Late CIA is characterized by an increase in fibrosis. Fibroblasts taken from RA patients with chronic disease are in a constitutive state of activation and exhibit plasticity in cell growth. Of the eight annotated genes that are selectively upregulated in late disease listed in cluster B of Table 1, four are involved in cell proliferation, differentiation and tumorigenesis and may play a role in the chronic activation of fibroblasts at late stages of disease. Specifically, tumor associated calcium signal transducer 2 is expressed early in tumorigenesis, and angiopoietin related protein 2 is associated with endothelial cell development and tumorigenesis.

### Example 7

### Genes Expressed in Early CIA

Several genes involved in cell proliferation, differentiation and tumorigenesis are selectively upregulated in early disease and are listed in cluster C of Table 1. CDC28 kinase binds to the catalytic subunit of cyclin dependent kinases and may be associated with dysregulation of lymphocyte cell cycle control in HIV infected patients. ADAM9, a disintegrin and metalloproteinase domain 9, binds MAD2beta, which is involved in cell cycle control.

Three apoptosis genes that are selectively upregulated in early CIA have anti-apoptotic properties. These include CD53, fibrinogen/angiopoietin related protein and baculoviral IAP repeat containing 2. The latter two are involved in endothelial cell survival. The upregulation of genes involved in endothelial cell survival, particularly early in disease, may allow for migration of inflammatory cells into the diseased joint.

Genes selectively upregulated in early arthritis (cluster C) include many inflammatory genes previously associated with CIA or RA. In addition, numerous other potentially pro-inflammatory genes are in this category. Pentaxin-related gene is involved in inflammatory reactions, particularly those of the vessel wall. Small inducible cytokine B subfamily member 13 (CXCL13) is a chemokine for B lymphocytes. Type II transmembrane protein is expressed exclusively in macrophages and monocytes and is involved in activation of myeloid cells. Hypoxia induced gene 2 (interleukin-20) is modulated by hypoxia and may have a role in inflammation, possibly in attempting to re-establish homeostasis.

### Example 8

### Genes that are Down-Regulated

Although most of the differentially-expressed genes were upregulated during CIA, all the genes in cluster A of Table 1 were downregulated, compared to normal paws. This represents a group of potentially important genes, as their downregulation may contribute to the loss of homeostasis in the joint and the failure to limit the inflammatory process. One annotated gene in cluster A, cytochrome P450, has previously been shown to be downregulated in inflammation and certain alleles of cytochrome P450, which are inactive or poor metabolizers, show a modest association with susceptibility to ankylosing spondylitis, but not RA. Most of the genes in cluster A are ESTs, and their further characterization will be of interest. In addition to the 25 ESTs in cluster A, the further characterization of the other 132 ESTs identified in this study will provide information about the gene regulatory network(s) involved in the autoimmune arthritic process.

In summary, the present study utilized DNA microarray technology to analyze coordinated gene expression in paws of mice with early and late CIA. This analysis has revealed a large number of genes previously not known to be involved in arthritis, as well as distinct gene expression profiles that differentiate between early and late CIA. Further characterization of these genes and pathways will advance the understanding of the basic mechanisms responsible for initiation and persistence of synovitis and may aid in the development of novel therapies.

### Example 9

### Isolation of Full-Length Genes Identified by ESTs

The 157 expressed sequence tags (ESTs) are used to identify the full-length genes associated with them. The EST sequences are used to search public and proprietary computer databases. Those that are not identified in the databases, are used to screen mouse libraries for full-length cDNA clones using methods known to one of skill in the art.

### Example 10

### Identification of Human Homologs and Production of a Human Microarray

Human homologs are identified by searching databases to find the closest human homolog for each of the 385 mouse genes identified herein. Many of the human homologs are known. Those that do not possess a homolog in the databases are identified by screening a human cDNA library using a mouse probe. In particular, when active regions or highly conserved regions of the mouse protein are known, these are used to screen the library. For example, kinases are known to contain regions that are highly conserved. Thus, if the mouse gene codes for a kinase, these regions are included within the probe. Alternatively, or in addition, a degenerate mouse probe is produced, with the degeneracy in regions that are less likely to possess high homology, for example, a degenerate probe for a kinase is constructed to have more degeneracy around the kinase region.

### Example 11

### mRNA Expression Profiling of Early and Late Rheumatoid Arthritis in Humans

Differential gene expression in the synovial tissue of humans with rheumatoid arthritis was analyzed and compared to that of synovial tissue from normal humans.

RNA was isolated from a human synovial biopsy and quick frozen in liquid nitrogen for storage at −80° C. Frozen synovial tissue was minced with a scalpel and homogenized with a Polytron Tissue Tearor (Biospec Products, Bartlesville, Okla.) in appropriate volumes of RNA Stat-60 (Tel-Test, Friendswood, Tex.). Total RNA was extracted from the tissue homogenates according to the manufacturer's instructions. Pooled total RNA from normal synovial biopsy samples, mild arthritic synovial biopsy samples and severe arthritic synovial biopsy samples was used to isolate polyA+ RNA using the Oligotex mRNA isolation kit (Qiagen, Valencia, Calif.) according to the manufacturer's instructions. RNA concentrations were measured by fluorometry using the Ribogreen RNA Quantification Kit (Molecular Probes, Inc., Eugene, Oreg.).

DNA microarray analysis was performed as follows: mRNA from a human without RA was used for normalization of gene expression levels across all microarray chips. Competitive hybridizations with Cy3 labeled normal human mRNA versus Cy5 labeled mild RA mRNA or Cy5 labeled severe RA mRNA were performed. Each sample (normal, mild and severe) was labeled and hybridized to the GeneChip® Human Genome U95 Set from Affymetrix (Santa Clara, Calif.) which represents about 60,000 full-length genes and EST clusters.

Primary data is examined using Incyte Gemtools software and GeneSpring version 4.0.4 software (Silicon Genetics, Redwood City, Calif.). Defective cDNA spots (irregular geometry, scratched, or <40% area compared to average) or spot fluorescence hybridizations with signal to noise ratios less than 2.5:1 are eliminated from the data set. Data sets are subjected to normalization first within each microarray experiment such that the median of the Cy5 channel was balanced against the ratio of the Cy3 channel (k*(MedianCy3)=MedianCy5, where k is the ratio of the median intensities in each). Each microarray contained 192 control genes present as non-mammalian single gene “spikes” or “complex targets”. The complex targets consist of probe-sets that contain a pool of cellular genes expressed in most cell types. In addition, each experimental mRNA sample was augmented with incremental amounts of non-mammalian gene RNA (2×, 4×, 16×, etc) to permit assessment of the dynamic range attained within each microarray. Little variation was observed across the microarray series with respect to the control genes (not shown), providing support for inter-array comparisons of temporally regulated genes. Genes were clustered according to their expression pattern by subjecting the log-transformed data (R=log₂Cy5/(kCy3), where R is the log of the expression ratio for each gene) to the hierarchical tree clustering algorithm as implemented in the GeneSpring program (Silicon Genetics). The hierarchical tree analysis was performed using a minimum distance value of 0.001, separation ratio of 0.5 and the standard correlation distance definition.

Human sense and antisense RNA probes were synthesized using the RNA Transcription Kit (Stratagene, La Jolla, Calif.). T3 or T7 RNA polymerase produced ³⁵S-radiolabeled antisense or sense single-stranded RNA probes, respectively. A sense probe generated from an unrelated human gene was used as a negative control for in situ hybridization.

For mild and severe disease, mRNA from patients with severe arthritis (score of 4) were used to generate probes that are hybridized to the GeneChip® Human Genome U95 Set from Affymetrix (Santa Clara, Calif.) which represents about 60,000 full-length genes and EST clusters, as is mRNA from normal human synovial tissue. Hybridizations are conducted on duplicate chips, allowing for the elimination of genes whose expression levels differed by greater than 50% between the duplicate samples. About 60,000 genes and ESTs are represented in the Set.

The method above seeks to identify all genes that are differentially expressed in human arthritis using a variety of microarrays or DNA chips. Using the information identified in Examples 9-11 a “human Rheumatoid Arthritis genechip” is produced.

### Example 12

### Method for the Production of a “Human Rheumatoid Arthritis Genechip”

The genes that are found to be differentially expressed in Examples 9-11 are used to produce a “human Rheumatoid Arthritis genechip.” This chip will be used for the diagnosis, prognosis, and treatment of the disease.

Other chips are produced with those differentially expressed genes that are only expressed in mild disease, a “mild RA” chip and those that are only differentially expressed in severe disease, a “severe RA” chip.

### Example 13

### Method for the Diagnosis and Staging of RA

mRNA is isolated from human synovial tissue, blood and human synovial fluid and treated as in Example 2. The microarray produced in Example 12 is analyzed for gene expression. From the analysis of up-and down-regulated genes a diagnosis and analysis of disease is made. The patient is monitored periodically during active disease and/or treatment. A prognosis is made based on these results as to the severity and chronic nature of the disease as well as the speed of deformity.

### Example 14

### Treatment of RA by Inhibiting Expression of Up-Regulated Genes

One or more of the genes that are up-regulated in Examples 4-6 are inhibited using antisense oligonucleotides or triple helix oligonucleotides. The antisense oligonucleotides are produced using methods known to one of skill in the art. The antisense oligonucleotides are administered intravenously, intramuscularly, or within a joint and the symptoms and disease is monitored.

### Example 15

### Treatment of RA by Activating Expression of Down-Regulated Genes

One or more of the genes that are down-regulated in Example 7 are activated using known transcriptional activators. Alternatively, expression vectors are administered that are targeted to the synovia and express one or more of the genes that are down-regulated. Preferably, the expression vectors are retroviral and are administered intravenously. The transcriptional activators and vectors are produced using methods known to one of skill in the art.

### Example 16

### Treatment of RA by Administration of Down-Regulated Proteins

One or more of the proteins that are down-regulated in Example 7 are purified and administered. The proteins are administered intravenously or into the joint.

### Example 17

### Use of Fibrinogen/Angiopoietin-Related Protein to Enhance Angiogenesis in Synovial Tissues and to Define the Involvement in Arthritic Processes

Because primers for fibrinogen/angiopoietin-related protein amplified a 270 base pair product from cDNA synthesized from mRNA from synovial tissues of RA patients, this suggests that this protein is involved in some way in the pathogenic process. Thus, expression of fibrinogen/angiopoietin-related protein is analyzed in various forms of RA and in situ in synovial tissue. If over-expression is identified in the process, anti-sense oligonucleotides are used to inhibit expression of fibrinogen/angiopoietin-related protein in synovia or systemically in the RA patients.

### Example 18

### Determination of the Best Treatment for a Patient with RA

From the results of the gene expression analysis, the best treatment for the patients with RA is determined. The treatment is based on the specific gene expression profile.

Thus, synovial fluid from a patient with rheumatoid arthritis is analyzed using a microarray as in Example 2. The analysis is used to identify the genes that are specifically up-regulated or down-regulated in that patient. Then, the treatment is selected based on the specific gene expression.

Although described in the context of certain preferred embodiments, the skilled artisan will appreciate that various changes and modifications can be made to the preferred embodiments, and such changes and modifications are meant to be encompassed by the invention, as defined by the appended claims.

### Example 19

### Correlation of mRNA Overexpression in CIA with Human Gene and Function: FARP

Microarray analyses identified fibrinogen/angiopoietin related protein (FARP) as one of the most highly over-expressed mRNAs (8734 tested) in arthritic paws of mice with collagen-induced arthritis (CIA). See Table 1, Cluster C, Mouse # W13905; Fibrinogen/angiopoietin-related protein. Data also demonstrated that human FARP. Data also demonstrated that human FARP mRNA is expressed in rheumatoid arthritis (RA) synovium. FARP is highly homologous to angiogenic factors and inhibits apoptosis of vascular endothelial cells in vitro. In RA, an increase in blood vessel formation, or angiogenesis, is observed in synovial tissue. Endothelial cells lining blood vessels can provide nutrients for inflamed tissue, allow infiltration of inflammatory cells, and secrete inflammatory cytokines, all of which contribute to disease processes. The suppression of arthritis by angiogenic inhibitors in animal models, such as CIA, further demonstrates that angiogenesis is necessary for arthritis. Mouse FARP mRNA is highly expressed during early stages of CIA and human FARP mRNA is expressed in RA synovial tissue.

### Example 20

### Characterizing FARP Expression in CIA

Prior to the present invention, FARP had not been described in arthritis. Localization of the cells that produce FARP mRNA and protein within the joint permits analysis FARP's role in angiogenesis in CIA. The cell types producing FARP mRNA and protein are determined and the role of FARP protein expression as it relates to the mRNA expression during CIA is identified.

Determination of spatial expression of FARP mRNA during CIA. DBA/1 mice are immunized with collagen as described in Thornton, et al. (1999) Arthritis Rheum 42:1109-1118. Mice are sacrificed 21, 28, 35, 42 and 49 days following primary collagen immunization. In situ hybridization analysis of FARP mRNA expression using sense and antisense probes generated from the FARP mouse cDNA are performed on tissue sections from paws of normal, unimmunized mice and arthritic mice.

Generation of antibody to FARP. An anti-FARP antibody is generated as described in Kim I, et al, (2000) Biochem J 346:603-610, and used for immunodetection and blocking of FARP function. Nucleotides 298 to 866 of the cDNA coding for the mouse FARP protein are cloned into the mammalian expression vector pcDNA3.1/His, which incorporates a histidine tag for easy isolation of the recombinant protein (Invitrogen, Carlsbad, Calif.). Following purification, this protein fragment encoding amino acids 100-289 of mouse FARP is injected into rabbits and serum is collected. Polyclonal antibody is purified from rabbit serum by ammonium sulfate precipitation and protein A column chromatography as described in Harlow E, et al, (1988) Antibodies: A laboratory manual. Cold Spring Harbor, N.Y., Cold Spring Harbor Laboratory; and Shanley J D, et al, (1994) J Infect Dis 169:1088-1091.

Determination of spatial and temporal expression of FARP protein during CIA. Since protein levels do not always directly reflect mRNA levels of a gene, the protein expression of FARP is determined in arthritic CIA paws using the anti-mouse FARP polyclonal antibody generated above. FARP protein is localized immunohistochemically using a horseradish peroxidase conjugated anti-rabbit secondary antibody. Sections are processed from paws of non-immunized mice and from paws of mice sacrificed 21, 28, 35, 42 and 49 days following primary collagen injection. Sera from non-immunized rabbits are used as a negative control. Sections from mouse liver are used as a positive control for immunohistochemical staining.

Results. In situ mRNA analysis demonstrates expression of FARP mRNA in the inflamed area of arthritic paws. FARP mRNA and protein are seen to be more highly expressed early in disease. In some embodiments, FARP protein is localized to the vasculature in arthritic paws. Blood vessel formation in CIA paws is readily observed by standard hematoxylin and eosin staining. However, co-localization of vasculature and FARP expression is demonstrated by analysis of serial sections for expression of endothelial cell-specific markers, such as von Willebrand factor Lu J, et al, (2000) J Immunol 164:5922-5927, in conjunction with FARP expression. The anti-human FARP polyclonal Ab from Kim, et. al. will be obtained, as this antibody will likely crossreact with mouse FARP. The homologous portion of mouse FARP protein that was previously used by Kim, et. al., is used to generate anti-human FARP polyclonal antibodies. Successful use of this polyclonal antibody in immunohistochemical staining demonstrates that administration of this portion of the protein to rabbits can generate polyclonal antibody to FARP. Polyclonal antibodies are easier and faster to generate than monoclonal antibodies; in some embodiments, the use of an antibody to block FARP function involves generation of a monoclonal antibody.

### Example 21

### Determining the Anti-Apoptotic Effects of FARP on Endothelial Cells

The angiogenic protein Ang1 and FARP have anti-apoptotic effects on endothelial cells. Ang1 mediates its anti-apoptotic effects by activating Tie2, an endothelial cell-specific receptor, resulting in phosphorylation of the serine-threonine kinase, Akt (protein kinase B) and mRNA upregulation of the apoptosis inhibitor, survivin. Papapetropoulos A, et al, (2000) J Biol Chem 275:9102-9105. FARP does not bind Tie2, but is highly homologous to Ang1 and is a secreted protein with anti-apoptotic effects on endothelial cells. FARP also has anti-apoptotic effects specific for endothelial cells, and is a secreted protein. Activation by FARP of an endothelial cell-specific receptor is found to result in the phosphorylation of specific anti-apoptotic intracellular molecules and increases mRNA expression of anti-apoptotic factors. Determination of the pathway that FARP utilizes in prolonging endothelial cell survival provides potential targets for therapeutic intervention. The effects of FARP on anti-apoptotic factors potentially regulating endothelial cell survival is identified.

In preferred embodiments, treatments and drug candidates that interfere with receptor binding by FARP lead to deactivation of the anti-apoptotic serine-threonine kinase, Akt, in endothelial cells. In further preferred embodiments, interference with expression of FARP, normal function of its receptor, and/or binding of FARP to its receptor also leads to decreased expression of survivin, Bcl2, and other anti-apoptotic factors in endothelial cells. Overall, these effects result in enhanced or normalized apoptosis of vascular endothelial cells in the arthritic joint, leading to a diminution or reversal of disease symptoms.

Expression and purification of recombinant mouse FARP (rmFARP). The entire cDNA coding for mouse FARP is inserted into the mammalian expression vector pcDNA3.1/His, which contains a six amino acid histidine tag for easy isolation of the protein (Invitrogen). The cDNA is transfected into COS-7 cells and purified from the cell supernatant. The anti-mouse FARP polyclonal antibody discussed above is used in Western blots to determine whether rmFARP is expressed in COS-7 cells.

Effects of FARP on endothelial cell expression of anti-apoptotic molecules. HUVEC (ATCC, Rockville, Md.) is treated with rmFARP in a range of 50 to 500 ng/ml as described for Ang1 (Papapetropoulos A, et al, (2000) J Biol Chem 275:9102-9105) and or with vehicle. RNA from these cells is analyzed by RNase protection assays (BD Pharmingen, San Diego, Calif.) for expression of the anti-apoptotic genes, survivin and Bcl-2, as previously performed in Thornton S, et al, (1999) Arthritis Rheum 42:1109-1118.

Effects of rmFARP administration on phosphorylation of serine-threonine kinases important in cell survival. Phosphorylation of the Akt survival serine threonine kinase is assessed as described in Papapetropoulos A, et al. Microvascular endothelial cells (Vec Technologies, Rensselaer, N.Y.) are treated with and without rmFARP. Anti-Akt antibody (Santa Cruz Biotechnology, Inc., Santa Cruz, Calif.) and phosphospecific Akt antibody (New England Biolabs, Beverly, Mass.) are used in Western blots to determine the amount of Akt protein present and the extent of Akt phosphorylation in these cells.

Results. rmFARP is found to increase the expression of survivin or Bcl-2 in endothelial cells, and also increases the phosphorylation of Akt. FARP is found to utilize a separate signaling pathway from Ang1, and other signaling molecules are thus analyzed for their role in the anti-apoptotic effects mediated by FARP. Additionally the anti-apoptotic molecules XIAP, c-IAP2 and NIAP are analyzed at the same time as survivin and Bcl-2 in the RNase protection analysis. These studies elucidate FARP's downstream effects that are mediated by a receptor.

### Example 22

### Determining the Role of FARP During CIA

Since FARP is one of the most highly overexpressed genes in CIA, and since it is also expressed in rheumatoid arthritis synovial tissue, its role in arthritis is tested both by administration and depletion of FARP before disease onset and during disease progression. In some embodiments, FARP aids in endothelial cell survival, allowing for increased inflammation in CIA. Thus, treatment with FARP can exacerbate CIA, and depletion of FARP can inhibit CIA. Recombinant mouse FARP, as well as antibodies to FARP, are administered before and during disease.

Effects of administration of rmFARP on the development and severity of CIA. rmFARP is administered i.p. to DBA/1 mice immunized with collagen. Based on published studies with other molecules (Thornton S, et al, (2000) J Immunol 165:1557-1563), FARP (10 ug/0.5 ml/mouse) is administered twice daily from days 14 to 21 following primary collagen immunization for testing effects before disease onset. For established disease, FARP is administered twice daily for seven days starting 24 hours after disease onset. Mice are scored daily for macroscopic signs of arthritis as described in Thornton S, et al, (1999) Arthritis Rheum 42:1109-1118. Mice are sacrificed at day 49 of disease and sections from treated and untreated mouse paws are analyzed histochemically for blood vessel formation and inflammatory cell infiltration by hematoxylin and eosin staining.

Effects of depletion of FARP on endothelial cell apoptosis. Antibody produced as described herein is used. Assessment of the ability of anti-FARP antibody to block the anti-apoptotic effects of FARP is performed in vitro with endothelial cell lines as described in Kim I, et al, (2000) Biochem J 346 Pt 3:603-610. Induction of apoptosis in HUVEC cells is performed by serum deprivation. HUVEC cells are grown for 24 hours in the presence of 10% serum and then incubated for 24 hours with the same media, or serum-free media with control buffer, rmFARP (200 and 800 ng/ml) or rmFARP plus anti-FARP antibody at varying concentrations. Analysis of apoptotic cells is as described in Kim, et al. Sera from unimmunized rabbits is used as a negative control.

Effects of depletion of FARP on the development and severity of CIA. Anti-FARP antibody is administered similarly to studies using anti-VEGF antibody in CIA (Sone H, et al, (2001) Biochem Biophys Res Commun 281:562-568). Antibody is delivered i.p. (200 ug/0.2 ml/mouse) every other day for 8 days both before (days 14-22) and during disease (24 hours after onset) as described above. Normal rabbit immunoglobulin and PBS are used as negative controls. Mice immunized with collagen are analyzed macroscopically and histologically as described above.

Results. It is found that administration of FARP protein to mice before disease onset can hasten the onset of disease, and that administration after disease onset can exacerbate disease symptoms and increase vasculature in the inflamed paws. Thus, in preferred embodiments, FARP is deleted by antibody. In alternative embodiments, a FARP knockout in DBA/1 mice is generated. Additionally, since FARP mRNA is synthesized in the rat embryo, it is implicated in embryonic development. In preferred embodiments, the antibody produced as described herein can block or interfere with the function of FARP. A polyclonal antibody produced in rabbits is optimized by using an affinity column made of the recombinant protein to purify the antibody. An alternative approach is to generate a monoclonal antibody. An advantage of using anti-FARP antibodies is the benefit of an antibody as a therapeutic agent.

### Example 23

### Involvement of FARP in Angiogenesis

FARP mRNA and protein are localized to the vascular endothelium in arthritic paws of CIA mice. Study of protein levels in such mice indicates that FARP protein levels correlate with FARP mRNA levels. Cells expressing FARP mRNA and protein during CIA are identified, and the kinetics of expression of FARP protein during CIA permits design of therapies and testing of candidate drugs having a specific and localized action on FARP mRNA and protein. Preferred therapies and drugs result in enhanced or normalized apoptosis of vascular endothelial cells in the arthritic joint, leading to a diminution or reversal of disease symptoms.

1. A method for the diagnosis and analysis of autoimmune disease or arthritides, in a patient, comprising:
obtaining a patient sample containing mRNA;
analyzing gene expression using the mRNA that results in a gene expression signature of that mRNA, wherein said gene expression signature comprises the identification and quantitation of gene expression from genes that have been identified as being differentially expressed in RA; and
using that gene expression signature to diagnose or analyze the autoimmune disease or arthritide in said patient, wherein said gene expression of at least about 60% of said genes correlates with that of said gene signature.

2. The method of claim 1 wherein said autoimmune disease or arthritides are selected from the group consisting of: Rheumatoid Arthritis, Lupus, Ankylosing Spondylitis, fibrositis, fibromyalgia, osteoarthritis, Gout, Juvenile Rheumatoid Arthritis, and an autoimmune disease caused by an infectious agent.

3. The method of claim 1 wherein said autoimmune disease or arthritide is rheumatoid arthritis.

4. The method of claim 1 wherein said patient is selected from the group consisting of: a human, a primate, a dog, a cat, a horse, and a sheep.

5. The method of claim 1, wherein said analysis is selected from the group consisting of: an analysis of severity of the disease, an analysis of pain manifestation, an analysis of deformity, an analysis of treatment methods, and an analysis of treatment efficacy.

6. The method of claim 1 wherein said gene expression analysis involves at least about 10 genes that are identified as differentially expressed in arthritis.

7. The method of claim 1 wherein said gene expression analysis involves at least about 50 genes that are identified as differentially expressed in arthritis.

8. The method of claim 1 wherein said gene expression analysis involves at least about 100 genes that are identified as differentially expressed in arthritis.

9. The method of claim 1, wherein said genes identified are expressed at least about 1.5 fold higher or lower than normal.

10. The method of claim 1, wherein said genes identified are expressed at least about 2 fold higher or lower than normal.

11. The method of claim 1, wherein said genes identified are expressed at least about 3 fold higher or lower than normal.

12. The method of claim 1, wherein said genes are selected from the group consisting of the 385 genes or ESTs in Table 1 (SEQ ID NOS: 1-385), homologs, or variant thereof.

13. The method of claim 1, wherein said genes are selected from the group consisting of:
the genes in cluster A.

14. The method of claim 13, wherein the genes in cluster A are down-regulated (SEQ ID NOS:1-37) at least about 2 fold.

15. The method of claim 1, wherein said genes are selected from the group consisting of: the genes in cluster B.

16. The method of claim 15, wherein the genes in cluster B are up-regulated (SEQ ID NOS:1-37) at least about 2 fold only in late or severe disease.

17. The method of claim 1, wherein said genes are selected from the group consisting of:
the genes in cluster C.

18. The method of claim 17, wherein the genes in cluster C are up-regulated (SEQ ID NOS:1-37) at least about 2 fold only in early or mild disease.

19. The method of claim 1, wherein said genes are selected from the group consisting of:
the genes in cluster D.

20. The method of claim 19, wherein the genes in cluster D are up-regulated (SEQ ID NOS:1-37) at least about 2 fold in early or mild disease and more in late or severe disease.

21. The method of claim 1, wherein said genes are selected from the group consisting of:
the genes in cluster E.

22. The method of claim 21, wherein the genes in cluster E are up-regulated (SEQ ID NOS:1-37) at least about 2 fold in both early or mile and late or severe disease.

23. The method of claim 1 wherein said differentially expressed genes are the 385 genes identified as SEQ ID NOS:1-385.

24. The method of claim 1 wherein if the genes in clusters B or D are upregulated, the disease is diagnosed as severe.

25. The method of claim 1 wherein if the genes in cluster A are upregulated, the disease is diagnosed as moderate to low-grade.

26. The method of claim 1, wherein said gene expression of at least about 70% of said genes correlates with that of said gene signature.

27. The method of claim 1, wherein said gene expression of at least about 80% of said genes correlates with that of said gene signature.

28. The method of claim 1, wherein said gene expression of at least about 90% of said genes correlates with that of said gene signature.

29. The method of claim 1, wherein said gene expression of at least about 95% of said genes correlates with that of said gene signature.

30. A method for the treatment of RA comprising:
down-regulating at least one of the genes identified in clusters B through D.

31. The method of claim 30 wherein said down-regulation is by adding antisense oligonucleotides specific for the gene that is being down-regulated.

32. The method of claim 30 wherein said down-regulation is by adding or expressing an repressor of the gene that is being down-regulated.

33. A method for the treatment of RA comprising:
up-regulating at least one of the genes in cluster A.

34. The method of claim 33 wherein said up-regulation is by adding or expressing a transcriptional activator of the gene that is being up-regulated.

35. The method of claim 33 wherein said up-regulation is by adding a vector that expresses the protein encoded by the gene that is being up-regulated.

36. A method for the identification of genes for targeting in the treatment of rheumatoid arthritis in a mammal other than a mouse, comprising:
identifying homologs of SEQ ID NOS:1-385.

37. A method for the diagnosis of rheumatoid arthritis in a mammal, comprising
obtaining a tissue or fluid sample from a diseased patient;
isolating mRNA from said sample;
using the isolated mRNA to analyze the gene expression of at least about 40 genes, selected from the group consisting of SEQ ID NOS:1-385 or a homolog thereof, obtaining a fingerprint of the patient's gene expression;
identifying whether at least about 60% of said fingerprint is at least about 2 fold differentially expressed from that of a normal patient.

38. An array or a genechip, specific for rheumatoid arthritis, comprising at least 10 of the genes selected from the group consisting of SEQ ID NOS:1-385 or homologs thereof.

39. The array or genechip of claim 38, comprising at least 40 of the genes selected from the group consisting of SEQ ID NOS:1-385 or homologs thereof.

40. The array or genechip of claim 38, comprising at least 50 of the genes selected from the group consisting of SEQ ID NOS:1 -385 or homologs thereof.

41. The array or genechip of claim 38, comprising at least 75 of the genes selected from the group consisting of SEQ ID NOS:1-385 or homologs thereof.

42. The array or genechip of claim 38, comprising at least 100 of the genes selected from the group consisting of SEQ ID NOS:1-385 or homologs thereof.

43. An array or a genechip, specific for rheumatoid arthritis consisting essentially of, at least 10 of the genes selected from the group consisting of SEQ ID NOS:1-385 or homologs thereof.

44. The array or genechip of claim 43, consisting essentially of at least 40 of the genes selected from the group consisting of SEQ ID NOS: 1-385.

45. The array or genechip of claim 43, consisting essentially of SEQ ID NOS:1-385.

46. The array or genechip of claim 38, wherein said genes allow for the identification of the severity of the disease.

47. The array or genechip of claim 38, wherein said genes allow for the prognosis of the disease.

48. The array or genechip of claim 38, wherein said genes allow for the diagnosis of the disease.

49. The array or genechip of claim 38, wherein said genes allow for the identification of the most efficacious treatment of the disease in a specific patient.

50. A method for the diagnosis or analyses of autoimmune disease or rheumatoid arthritis, comprising
obtaining mRNA from a patient;
using the mRNA as a probe for the analysis of the array or genechip of claim 38;
comparing the results obtained with those of a normal patient.

51. A method of screening the efficacy of a candidate drug in vitro for the treatment of collagen-induced arthritis comprising:
identifying vascular endothelial cells expressing FARP mRNA and protein;
introducing a candidate drug to said endothelial cells; and
evaluating whether said candidate drug causes enhanced or normalized apoptosis of vascular endothelial cells.

52. A method of reducing the symptoms associated with collagen-induced arthritis comprising:
identifying a subject suffering from collagen-induced arthritis; and
administering a compound effective to deplete at least one of the group of FARP mRNA, FARP protein, FARP receptor binding, and FARP activity.

53. The method of claim 52, wherein said compound is an anti-FARP antibody.

54. The methof of claim 53, wherein said antibody interferes with binding of FARP to a FARP receptor.
